(19) United States
(12) Patent Application Publication
(10) Pub. No.: US 2022/0202861 A1
(43) Pub. Date: Jun. 30, 2022

(54) TREATMENT OF LIVER FAILURE BY EX VIVO REPROGRAMMED IMMUNE CELLS

(71) Applicant: CREATIVE MEDICAL TECHNOLOGIES, INC., Phoenix, AZ (US)

(72) Inventors: Thomas Ichim, San Diego, CA (US); Amit Patel, Salt Lake City, UT (US); Timothy Warbington, Phoenix, AZ (US)

(73) Assignee: CREATIVE MEDICAL TECHNOLOGIES, INC., Phoenix, AZ (US)

(21) Appl. No.: 17/559,985

(22) Filed: Dec. 22, 2021

Related U.S. Application Data
(60) Provisional application No. 63/131,261, filed on Dec. 28, 2020.

(51) Int. Cl. A61K 35/17; A61P 1/16; C12N 5/0735

(52) U.S. Cl. CPC A61K 35/17; C12N 5/0606; A61P 1/16

(57) ABSTRACT

Disclosed are methods, means and compositions of matter useful for treatment of liver failure using ex vivo reprogrammed immune cells. In one embodiment, cells of the recipient (autologous) are cocultured with a regenerative cell population alone or in the presence of one or more adjuvants. Said adjuvants enhance transfer of regenerative activity from said mesenchymal stem cells to said immune cells. In one embodiment said ex vivo reprogrammed immune cells are capable of inducing death or inactivation of hepatic stellate cells. In other embodiments, said immune cells provide antifibrotic activity to induce suppression of liver cirrhosis. In other embodiments, said immune cells provide for growth factors to enhance hepatic regeneration.

### CROSS-REFERENCE TO RELATED APPLICATIONS

This application claims priority to U.S. Provisional Application No. 63/131,261, filed Dec. 28, 2020, which is herein incorporated by reference in its entirety.

### FIELD OF THE INVENTION

The invention pertains to the field of liver failure, more specifically the invention relates to treatment of liver failure with immune modulatory cells, more specifically, the invention relates to treatment of liver failure with ex vivo stem cell reprogrammed cells.

### BACKGROUND OF THE INVENTION

It is widely recognized that liver failure is a serious and if untreated, fatal medical condition that occurs as a result of a number of acute and chronic clinical inciting factors, including drug/alcohol-induced hepatotoxicity, viral infections, vascular injury, autoimmune disease, or genetic predisposition [1]. Manifestations of liver failure include fulminant acute hepatitis, chronic hepatitis, or cirrhosis. Subsequent to various acute insults to the liver, the organ regenerates due to its unique self-renewal activity. If the insult is continuously occurring, the liver's capacity to regenerate new cells is overwhelmed and fibrotic non-functional tissue is deposited which takes over the function of the hepatic parenchyma. The subsequent reduction of hepatocyte function can give rise to metabolic instability combined with disruption of essential bodily functions (i.e., energy supply, acid-base balance and coagulation) [2-4]. If not rapidly addressed, complications of hepatic dysfunction such as uncontrolled bleeding and sepsis occur, and dependent organs such as the brain and kidneys cease to function because of accumulation of toxic metabolites [5]. In critical cases, such as when patients progress to Acute-to-Chronic Live Failure (ACLF), liver transplant is considered to be the standard treatment. However, there are often serious difficulties to obtain a suitable donor and many complications arise after transplantation, including rejection and long-term adherence to immunosuppressant regimes [6, 7].

Interest in regenerative approaches toward liver failure is associated not only by the unmet medical need for novel interventions, but also is based on the inherent ability of the liver to self-regenerate. It has been demonstrated that up to 70% resection of the liver results in complete regeneration [8, 9]. Clinically, the potent regenerative ability of the liver allows for procedures such as living donor transplantation, two-stage hepatectomies, and split liver transplantation, which would be impossible with other organs which do not possess the potent inherent regenerative properties of the liver [10-12]. Liver regeneration is a tightly regulated process that adapts to the specific size of the body, specifically, after partial hepatectomy, the liver will only regenerate to the original size, and not hypertrophy. An interesting example of the tight regulation of liver regeneration is seen in transplantation medicine where the donor liver adapts to the size of the recipient body. For example, smaller livers transplanted in proportionally larger recipients take on a larger size and vice versa [13, 14].

Mechanistically, the process of liver regeneration occurs in three broad phases: a) Priming; b) Proliferation and c) Termination [8]. It is important to note that hepatocytes are not terminally differentiated cells, but cells that reside in a state of proliferative quiescence. Specifically, they share features with other regenerative cells such hematopoietic stem cells, in that they are normally in the G0 phase of cell cycle. This is altered during liver regeneration, which is described below.

During the priming phase, numerous injury signals are generated as a result of the underlying injury, these include activators of toll like receptors, complement degradation products, and Damage Associated Molecular Patterns (DAMPs). These signals stimulate various cells, primarily Kupffer cells, to produce cytokines and growth factors such as IL-6, TNF-alpha, and HGF which induce entry of hepatocytes into cell cycle. The importance of these molecular signals in the initiation of liver regeneration is highlighted by knockout studies. Cressmann et al demonstrated in a partial hepatectomy IL-6 knockout model blockade of liver regeneration that was associated with blunted exit from G0 phase of cell cycle in hepatocytes of these mice but not in nonparenchymal liver cells. Furthermore, they conclusively showed the importance of IL-6 in that a single preoperative dose of recombinant IL-6 restored post-injury hepatocyte entry into G1/2 to levels observed in wild-type mice and restored biochemical function [15]. NF-kappa B is a major downstream effector of various inflammatory cytokines including TNF-alpha and IL-6. Melato et al generated hepatic specific knockout mice in which the inhibitor of NF-kappa B, IKK2, was ablated, thus giving rise to a higher level of background NF-kappa B activation. In these mice partial hepatectomy resulted in accelerated entry of hepatocytes into cell cycle [16]. The role of a variety of inflammatory or “danger” associated pathways in the initial priming of hepatocyte proliferation after injury has been confirmed using DNA microarray analysis of genes associated with these signaling pathways such as STAT, p38MAPK, and Ras/ERK [17].

The Proliferation Phase of hepatic regeneration is associated with “primed” hepatocytes leaving G₁ stage of cell cycle and entering S phase, which is accompanied by phosphorylation of the retinoblastoma protein (pRb) and by up-regulated expression of a number of proliferation associated genes including cyclin E, cyclin A, and DNA polymerase [18, 19]. Key cytokines involved in stimulation of proliferation of the hepatocytes include hepatocyte growth factor (HGF) and epidermal growth factor (EGF). HGF is produced by mesenchymal cells, hepatic stellate cells, and liver sinusoidal endothelial cells as a pro-protein, which acts both systemically and locally [20, 21]. Systemic elevations in HGF are observed after partial hepatectomy [22], whereas local HGF is released from its latent form which is often bound to extracellular matrix proteins [23]. Activation of HGF occurs typically via enzymatic cleavage mediated by urokinase type plasminogen activator (uPA) [24, 25]. The importance of HGF in the Proliferation Phase of liver regeneration is observed in animals where the HGF receptor c-MET is conditionally inactivated, which display a reduction in hepatocyte entry into the S phase of cell cycle post injury [26]. EGF signaling has also been demonstrated to be involved in entry into the proliferative phase post injury. Natarajan et al. performed perinatal deletion of EGFR in hepatocytes prior to partial hepatectomy. They showed that after hepatic injury mice lacking EGFR in the liver had an increased mortality accompanied by increased levels of serum transaminases indicating liver damage. Liver regeneration was delayed in the mutants because of reduced hepatocyte proliferation. Analysis of cell cycle progression in EGFR-deficient livers indicated a defective G(1)-S phase entry with delayed transcriptional activation and reduced protein expression of cyclin D1 followed by reduced cdk2 and cdk1 expression [27].

The Termination Phase of liver regeneration occurs when the normal liver-mass/body-weight ratio of 2.5% has been restored [28]. While in the Initiation Phase of liver regeneration, several inflammatory cytokines are critical, in the Termination Phase, antiinflammatory cytokines such as IL-10 [29], are upregulated, which dampen proliferative stimuli [30]. Additionally, cytokines with direct antiproliferative activity such as TGF-beta are generated, which result in cell cycle arrest of proliferating hepatocytes.

While classical liver regeneration is mediated by hepatocytes [8, 31] in certain situations, such as in liver failure, the ability of the hepatocytes to mediate regeneration is limited and liver progenitor cells (LPCs) must carry out the process. The concept of a LPC, which took over regenerative function when hepatocyte multiplication is stunted, was first demonstrated in 1956 when Farber treated rats with various liver carcinogens that blocked division of hepatocytes [32]. He discovered the existence of “Oval Cells” which were subsequently demonstrated to act as LPC having ability to differentiate into both hepatocytes and biliary cells [33]. LPC are found in the canals of Hering and bile ductules in human liver and found increased in patients with chronic liver disease [34]. It is unclear what the origin of LPCs is, whether they derive from local cells, or directly from MSCs[35], particularly bone marrow derived MSCs[36], but the cellular mechanisms are poorly understood[37]. In 2000 Theise et al [38] found hepatocytes and cholangiocytes derived from extrahepatic circulating stem cells in the livers of female patients who had undergone therapeutic bone marrow transplantations. In the two female recipients from male donors and four male recipients from female donors hepatocyte and cholangiocyte engraftment ranged from 4% to 43% and from 4% to 38%, respectively.

Given the potent regenerative nature of the liver, combined with the possibility that extrahepatic cellular sources may contribute to regeneration, numerous attempts have been made to utilize cellular therapy for treatment of liver failure. The original hepatic cellular therapies involved the administration of allogeneic hepatocytes, which was attempted in animal models more than 30 years ago and is experimentally used clinically. Unfortunately, major hurdles exist that block this procedures from routine use, specifically: a) low number of suitable donors; b) extremely poor hepatocyte viability after transplantation, with some groups as low as 30%; and c) need for continuous immune suppression which possesses inherent adverse effects [39].

### SUMMARY

Preferred embodiments herein include methods of preventing, and/or inhibiting, and/or reversing liver failure comprising the steps of: a) identifying a patient suffering from liver failure; b) extracting from said patient immune cells; c) contacting said immune cells with regenerative cells in a manner so that regenerative cells endow onto said immune cells properties capable of inhibiting and/or reversing liver failure; and d) administering said immune cells into said patient.

Preferred methods include embodiments wherein said liver failure is associated with fibrosis.

Preferred methods include embodiments wherein said liver failure is associated with alcoholism.

Preferred methods include embodiments wherein said liver failure is associated with viral damage.

Preferred methods include embodiments wherein said liver failure is associated with inflammation.

Preferred methods include embodiments wherein said liver failure is non-alcoholic steatohepatitis.

Preferred methods include embodiments wherein said liver failure is autoimmune mediated.

Preferred methods include embodiments wherein said immune cells are extracted from a patient who is not the recipient.

Preferred methods include embodiments wherein said immune cells are xenogeneic.

Preferred methods include embodiments wherein said immune cells are cord blood derived.

Preferred methods include embodiments wherein said immune cells are derived from pluripotent stem cells.

Preferred methods include embodiments wherein said immune cells are cultured together with said regenerative cells in the presence of an activator of an immune receptor.

Preferred methods include embodiments wherein said immune receptor activates immunotyrosine activation motifs.

Preferred methods include embodiments wherein said immune receptor activates NF-AT.

Preferred methods include embodiments wherein said immune receptor activates NF-kappa B.

Preferred methods include embodiments wherein said immune receptor activates STAT-3.

Preferred methods include embodiments wherein said immune receptor activates STAT-4.

Preferred methods include embodiments wherein said immune receptor activates janus activated kinase.

Preferred methods include embodiments wherein said immune receptor activates MAP-kinase.

Preferred methods include embodiments wherein said immune receptor is TLR. 1

Preferred methods include embodiments wherein said TLR-1 is activated by Pam3CSK4.

Preferred methods include embodiments wherein said immune receptor is TLR-2

Preferred methods include embodiments wherein said TLR-2 is activated by HKLM.

Preferred methods include embodiments wherein said immune receptor is TLR-3.

Preferred methods include embodiments wherein said TLR-3 is activated by Poly:IC.

Preferred methods include embodiments wherein said immune receptor is TLR-4.

Preferred methods include embodiments wherein said TLR-4 is activated by LPS.

Preferred methods include embodiments wherein said TLR-4 is activated by Buprenorphine.

Preferred methods include embodiments wherein said TLR-4 is activated by Carbamazepine.

Preferred methods include embodiments wherein said TLR-4 is activated by Fentanyl.

Preferred methods include embodiments wherein said TLR-4 is activated by Levorphanol.

Preferred methods include embodiments wherein said TLR-4 is activated by Methadone.

Preferred methods include embodiments wherein said TLR-4 is activated by Cocaine.

Preferred methods include embodiments wherein said TLR-4 is activated by Morphine.

Preferred methods include embodiments wherein said TLR-4 is activated by Oxcarbazepine.

Preferred methods include embodiments wherein said TLR-4 is activated by Oxycodone.

Preferred methods include embodiments wherein said TLR-4 is activated by Pethidine.

Preferred methods include embodiments wherein said TLR-4 is activated by Glucuronoxylomannan from Cryptococcus.

Preferred methods include embodiments wherein said TLR-4 is activated by Morphine-3-glucuronide.

Preferred methods include embodiments wherein said TLR-4 is activated by lipoteichoic acid.

Preferred methods include embodiments wherein said TLR-4 is activated by beta.-defensin 2.

Preferred methods include embodiments wherein said TLR-4 is activated by low molecular weight hyaluronic acid.

Preferred methods include embodiments wherein said low molecular weight hyaluronic acid has a molecular weight of <1000 kDa.

Preferred methods include embodiments wherein said low molecular weight hyaluronic acid has a molecular weight of <500 kDa.

Preferred methods include embodiments wherein said low molecular weight hyaluronic acid has a molecular weight of <250 kDa.

Preferred methods include embodiments wherein said low molecular weight hyaluronic acid has a molecular weight of <100 kDa.

Preferred methods include embodiments wherein said TLR-4 is activated by fibronectin EDA.

Preferred methods include embodiments, wherein said TLR-4 is activated by snapin.

Preferred methods include embodiments wherein said TLR-4 is activated by tenascin C.

Preferred methods include embodiments wherein said immune receptor is TLR-5.

Preferred methods include embodiments wherein said TLR-5 is activated by flaggelin.

Preferred methods include embodiments wherein said immune receptor is TLR-6.

Preferred methods include embodiments wherein said TLR-6 is activated by FSL-1.

Preferred methods include embodiments wherein said immune receptor is TLR-7.

Preferred methods include embodiments wherein said TLR-7 is activated by imiquimod.

Preferred methods include embodiments wherein said immune receptor is TLR-8.

Preferred methods include embodiments wherein said TLR-8 is activated by ssRNA40/LyoVec.

Preferred methods include embodiments wherein said immune receptor is TLR-9.

Preferred methods include embodiments wherein said TLR-9 is activated by a CpG oligonucleotide.

Preferred methods include embodiments wherein said TLR-9 is activated by ODN2006.

Preferred methods include embodiments wherein said TLR-9 is activated by Agatolimod.

Preferred methods include embodiments wherein said TLR-9 is activated by ODN2007.

Preferred methods include embodiments wherein said TLR-9 is activated by ODN1668.

Preferred methods include embodiments wherein said TLR-9 is activated by ODN1826.

Preferred methods include embodiments wherein said TLR-9 is activated by ODN BW006.

Preferred methods include embodiments wherein said TLR-9 is activated by ODN D SL01.

Preferred methods include embodiments wherein said TLR-9 is activated by ODN 2395.

Preferred methods include embodiments wherein said TLR-9 is activated by ODN M362.

Preferred methods include embodiments wherein said TLR-9 is activated by ODN SL03.

Preferred methods include embodiments wherein said regenerative cell is a stem cell.

Preferred methods include embodiments, wherein said stem cell is a hematopoietic stem cell.

Preferred methods include embodiments wherein said hematopoietic stem cell is capable of generating leukocytic, lymphocytic, thrombocytic and erythrocytic cells when transplanted into an immunodeficient animal.

Preferred methods include embodiments wherein said hematopoietic stem cell expresses interleukin-3 receptor.

Preferred methods include embodiments wherein said hematopoietic stem cell expresses interleukin-1 receptor.

Preferred methods include embodiments wherein said hematopoietic stem cell expresses c-met.

Preferred methods include embodiments wherein said hematopoietic stem cell expresses mpl.

Preferred methods include embodiments wherein said hematopoietic stem cell expresses interleukin-11 receptor.

Preferred methods include embodiments wherein said hematopoietic stem cell expresses G-CSF receptor.

Preferred methods include embodiments wherein said hematopoietic stem cell expresses GM-CSF receptor.

Preferred methods include embodiments wherein said hematopoietic stem cell expresses M-CSF receptor.

Preferred methods include embodiments wherein said hematopoietic stem cell expresses VEGF-receptor.

Preferred methods include embodiments wherein said hematopoietic stem cell expresses c-kit.

Preferred methods include embodiments wherein said hematopoietic stem cell expresses CD33.

Preferred methods include embodiments wherein said hematopoietic stem cell expresses CD133.

Preferred methods include embodiments wherein said hematopoietic stem cell expresses CD34.

Preferred methods include embodiments wherein said hematopoietic stem cell expresses Fas ligand.

Preferred methods include embodiments wherein said hematopoietic stem cell does not express lineage markers.

Preferred methods include embodiments wherein said hematopoietic stem cell does not express CD14.

Preferred methods include embodiments wherein said hematopoietic stem cell does not express CD16.

Preferred methods include embodiments wherein said hematopoietic stem cell does not express CD3.

Preferred methods include embodiments wherein said hematopoietic stem cell does not express CD56.

Preferred methods include embodiments wherein said hematopoietic stem cell does not express CD38.

Preferred methods include embodiments wherein said hematopoietic stem cell does not express CD30.

Preferred methods include embodiments wherein said regenerative cell is a mesenchymal stem cell.

Preferred methods include embodiments wherein said mesenchymal stem cells are naturally occurring mesenchymal stem cells.

Preferred methods include embodiments wherein said mesenchymal stem cells are generated in vitro.

Preferred methods include embodiments wherein said naturally occurring mesenchymal stem cells are tissue derived.

Preferred methods include embodiments wherein said naturally occurring mesenchymal stem cells are derived from a bodily fluid.

Preferred methods include embodiments wherein said tissue derived mesenchymal stem cells are selected from a group comprising of: a) bone marrow; b) perivascular tissue; c) adipose tissue; d) placental tissue; e) amniotic membrane; f) omentum; g) tooth; h) umbilical cord tissue; i) fallopian tube tissue; j) hepatic tissue; k) renal tissue; 1) cardiac tissue; m) tonsillar tissue; n) testicular tissue; o) ovarian tissue; p) neuronal tissue; q) auricular tissue; r) colonic tissue; s) submucosal tissue; t) hair follicle tissue; u) pancreatic tissue; v) skeletal muscle tissue; and w) subepithelial umbilical cord tissue.

Preferred methods include embodiments wherein said tissue derived mesenchymal stem cells are isolated from tissues containing cells selected from a group of cells comprising of: endothelial cells, epithelial cells, dermal cells, endodermal cells, mesodermal cells, fibroblasts, osteocytes, chondrocytes, natural killer cells, dendritic cells, hepatic cells, pancreatic cells, stromal cells, salivary gland mucous cells, salivary gland serous cells, von Ebner's gland cells, mammary gland cells, lacrimal gland cells, ceruminous gland cells, eccrine sweat gland dark cells, eccrine sweat gland clear cells, apocrine sweat gland cells, gland of Moll cells, sebaceous gland cells. bowman's gland cells, Brunner's gland cells, seminal vesicle cells, prostate gland cells, bulbourethral gland cells, Bartholin's gland cells, gland of Littre cells, uterus endometrium cells, isolated goblet cells, stomach lining mucous cells, gastric gland zymogenic cells, gastric gland oxyntic cells, pancreatic acinar cells, paneth cells, type II pneumocytes, clara cells, somatotropes, lactotropes, thyrotropes, gonadotropes, corticotropes, intermediate pituitary cells, magnocellular neurosecretory cells, gut cells, respiratory tract cells, thyroid epithelial cells, parafollicular cells, parathyroid gland cells, parathyroid chief cell, oxyphil cell, adrenal gland cells, chromaffin cells, Leydig cells, theca interna cells, corpus luteum cells, granulosa lutein cells, theca lutein cells, juxtaglomerular cell, macula densa cells, peripolar cells, mesangial cell, blood vessel and lymphatic vascular endothelial fenestrated cells, blood vessel and lymphatic vascular endothelial continuous cells, blood vessel and lymphatic vascular endothelial splenic cells, synovial cells, serosal cell (lining peritoneal, pleural, and pericardial cavities), squamous cells, columnar cells, dark cells, vestibular membrane cell (lining endolymphatic space of ear), stria vascularis basal cells, stria vascularis marginal cell (lining endolymphatic space of ear), cells of Claudius, cells of Boettcher, choroid plexus cells, pia-arachnoid squamous cells, pigmented ciliary epithelium cells, nonpigmented ciliary epithelium cells, corneal endothelial cells, peg cells, respiratory tract ciliated cells, oviduct ciliated cell, uterine endometrial ciliated cells, rete testis ciliated cells, ductulus efferens ciliated cells, ciliated ependymal cells, epidermal keratinocytes, epidermal basal cells, keratinocyte of fingernails and toenails, nail bed basal cells, medullary hair shaft cells, cortical hair shaft cells, cuticular hair shaft cells, cuticular hair root sheath cells, hair root sheath cells of Huxley's layer, hair root sheath cells of Henle's layer, external hair root sheath cells, hair matrix cells, surface epithelial cells of stratified squamous epithelium, basal cell of epithelia, urinary epithelium cells, auditory inner hair cells of organ of Corti, auditory outer hair cells of organ of Corti, basal cells of olfactory epithelium, cold-sensitive primary sensory neurons, heat-sensitive primary sensory neurons, Merkel cells of epidermis, olfactory receptor neurons, pain-sensitive primary sensory neurons, photoreceptor rod cells, photoreceptor blue-sensitive cone cells, photoreceptor green-sensitive cone cells, photoreceptor red-sensitive cone cells, proprioceptive primary sensory neurons, touch-sensitive primary sensory neurons, type I carotid body cells, type II carotid body cell (blood pH sensor), type I hair cell of vestibular apparatus of ear (acceleration and gravity), type II hair cells of vestibular apparatus of ear, type I taste bud cells cholinergic neural cells, adrenergic neural cells, peptidergic neural cells, inner pillar cells of organ of Corti, outer pillar cells of organ of Corti, inner phalangeal cells of organ of Corti, outer phalangeal cells of organ of Corti, border cells of organ of Corti, Hensen cells of organ of Corti, vestibular apparatus supporting cells, taste bud supporting cells, olfactory epithelium supporting cells, Schwann cells, satellite cells, enteric glial cells, astrocytes, neurons, oligodendrocytes, spindle neurons, anterior lens epithelial cells, crystallin-containing lens fiber cells, hepatocytes, adipocytes, white fat cells, brown fat cells, liver lipocytes, kidney glomerulus parietal cells, kidney glomerulus podocytes, kidney proximal tubule brush border cells, loop of Henle thin segment cells, kidney distal tubule cells, kidney collecting duct cells, type I pneumocytes, pancreatic duct cells, nonstriated duct cells, duct cells, intestinal brush border cells, exocrine gland striated duct cells, gall bladder epithelial cells, ductulus efferens nonciliated cells, epididymal principal cells, epididymal basal cells, ameloblast epithelial cells, planum semilunatum epithelial cells, organ of Corti interdental epithelial cells, loose connective tissue fibroblasts, corneal keratocytes, tendon fibroblasts, bone marrow reticular tissue fibroblasts, nonepithelial fibroblasts, pericytes, nucleus pulposus cells, cementoblast/cementocytes, odontoblasts, odontocytes, hyaline cartilage chondrocytes, fibrocartilage chondrocytes, elastic cartilage chondrocytes, osteoblasts, osteocytes, osteoclasts, osteoprogenitor cells, hyalocytes, stellate cells (ear), hepatic stellate cells (Ito cells), pancreatic stelle cells, red skeletal muscle cells, white skeletal muscle cells, intermediate skeletal muscle cells, nuclear bag cells of muscle spindle, nuclear chain cells of muscle spindle, satellite cells, ordinary heart muscle cells, nodal heart muscle cells, Purkinje fiber cells, smooth muscle cells, myoepithelial cells of iris, myoepithelial cell of exocrine glands, melanocytes, retinal pigmented epithelial cells, oogonia/oocytes, spermatids, spermatocytes, spermatogonium cells, spermatozoa, ovarian follicle cells, Sertoli cells, thymus epithelial cell, and/or interstitial kidney cells.

Preferred methods include embodiments wherein said mesenchymal stem cells are plastic adherent.

Preferred methods include embodiments wherein said mesenchymal stem cells express a marker selected from a group comprising of: a) CD73; b) CD90; and c) CD105.

Preferred methods include embodiments wherein said mesenchymal stem cells lack expression of a marker selected from a group comprising of: a) CD14; b) CD45; and c) CD34.

Preferred methods include embodiments wherein said mesenchymal stem cells from umbilical cord tissue express markers selected from a group comprising of; a) oxidized low density lipoprotein receptor 1, b) chemokine receptor ligand 3; and c) granulocyte chemotactic protein.

Preferred methods include embodiments wherein said mesenchymal stem cells from umbilical cord tissue do not express markers selected from a group comprising of: a) CD117; b) CD31; c) CD34; and CD45;

Preferred methods include embodiments wherein said mesenchymal stem cells from umbilical cord tissue express, relative to a human fibroblast, increased levels of interleukin 8 and reticulon 1

Preferred methods include embodiments wherein said mesenchymal stem cells from umbilical cord tissue have the potential to differentiate into cells of at least a skeletal muscle, vascular smooth muscle, pericyte or vascular endothelium phenotype.

Preferred methods include embodiments wherein said mesenchymal stem cells from umbilical cord tissue express markers selected from a group comprising of: a) CD10; b) CD13; c) CD44; d) CD73; and e) CD90.

Preferred methods include embodiments wherein said umbilical cord tissue mesenchymal stem cell is an isolated umbilical cord tissue cell isolated from umbilical cord tissue substantially free of blood that is capable of self-renewal and expansion in culture,

Preferred methods include embodiments wherein said umbilical cord tissue mesenchymal stem cells has the potential to differentiate into cells of other phenotypes.

Preferred methods include embodiments wherein said other phenotypes comprise: a) osteocytic; b) adipogenic; and c) chondrogenic differentiation.

Preferred methods include embodiments wherein said cord tissue derived mesenchymal stem cells can undergo at least 20 doublings in culture.

Preferred methods include embodiments wherein said cord tissue derived mesenchymal stem cell maintains a normal karyotype upon passaging

Preferred methods include embodiments wherein said cord tissue derived mesenchymal stem cell expresses a marker selected from a group of markers comprised of: a) CD10 b) CD13; c) CD44; d) CD73; e) CD90; f) PDGFr-alpha; g) PD-L2; and h) HLA-A,B,C

Preferred methods include embodiments wherein said cord tissue mesenchymal stem cells does not express one or more markers selected from a group comprising of; a) CD31; b) CD34; c) CD45; d) CD80; e) CD86; f) CD117; g) CD141; h) CD178; i) B7-H2; j) HLA-G and k) HLA-DR,DP,DQ.

Preferred methods include embodiments wherein said umbilical cord tissue-derived cell secretes factors selected from a group comprising of: a) MCP-1; b) MIP1beta; c) IL-6; d) IL-8; e) GCP-2; f) HGF; g) KGF; h) FGF; i) HB-EGF; j) BDNF; k) TPO; l) RANTES; and m) TIMP1

Preferred methods include embodiments wherein said umbilical cord tissue derived cells express markers selected from a group comprising of: a) TRA1-60; b) TRA1-81; c) SSEA3; d) SSEA4; and e) NANOG.

Preferred methods include embodiments wherein said umbilical cord tissue-derived cells are positive for alkaline phosphatase staining.

Preferred methods include embodiments wherein said umbilical cord tissue-derived cells are capable of differentiating into one or more lineages selected from a group comprising of; a) ectoderm; b) mesoderm, and; c) endoderm.

Preferred methods include embodiments wherein said bone marrow derived mesenchymal stem cells possess markers selected from a group comprising of: a) CD73; b) CD90; and c) CD105.

Preferred methods include embodiments wherein said bone marrow derived mesenchymal stem cells possess markers selected from a group comprising of: a) LFA-3; b) ICAM-1; c) PECAM-1; d) P-selectin; e) L-selectin; f) CD49b/CD29; g) CD49c/CD29; h) CD49d/CD29; i) CD29; j) CD18; k) CD61; 1) 6-19; m) thrombomodulin; n) telomerase; o) CD10; p) CD13; and q) integrin beta.

Preferred methods include embodiments wherein said bone marrow derived mesenchymal stem cell is a mesenchymal stem cell progenitor cell.

Preferred methods include embodiments wherein said mesenchymal progenitor cells are a population of bone marrow mesenchymal stem cells enriched for cells containing STRO-1

Preferred methods include embodiments wherein said mesenchymal progenitor cells express both STRO-1 and VCAM-1.

Preferred methods include embodiments wherein said STRO-1 expressing cells are negative for at least one marker selected from the group consisting of: a) CBFA-1; b) collagen type II; c) PPAR.gamma2; d) osteopontin; e) osteocalcin; f) parathyroid hormone receptor; g) leptin; h) H-ALBP; i) aggrecan; j) Ki67, and k) glycophorin A.

Preferred methods include embodiments wherein said bone marrow mesenchymal stem cells lack expression of CD14, CD34, and CD45.

Preferred methods include embodiments wherein said STRO-1 expressing cells are positive for a marker selected from a group comprising of: a) VACM-1; b) TKY-1; c) CD146 and; d) STRO-2

Preferred methods include embodiments wherein said bone marrow mesenchymal stem cell express markers selected from a group comprising of: a) CD13; b) CD34; c) CD56 and; d) CD117

Preferred methods include embodiments wherein said bone marrow mesenchymal stem cells do not express CD10.

Preferred methods include embodiments wherein said bone marrow mesenchymal stem cells do not express CD2, CD5, CD14, CD19, CD33, CD45, and DRII.

Preferred methods include embodiments wherein said bone marrow mesenchymal stem cells express CD13, CD34, CD56, CD90, CD117 and nestin, and which do not express CD2, CD3, CD10, CD14, CD16, CD31, CD33, CD45 and CD64.

Preferred methods include embodiments wherein said skeletal muscle stem cells express markers selected from a group comprising of: a) CD13; b) CD34; c) CD56 and; d) CD117

Preferred methods include embodiments wherein said skeletal muscle mesenchymal stem cells do not express CD10.

Preferred methods include embodiments wherein said skeletal muscle mesenchymal stem cells do not express CD2, CD5, CD14, CD19, CD33, CD45, and DRII.

Preferred methods include embodiments wherein said bone marrow mesenchymal stem cells express CD13, CD34, CD56, CD90, CD117 and nestin, and which do not express CD2, CD3, CD10, CD14, CD16, CD31, CD33, CD45 and CD64.

Preferred methods include embodiments wherein said subepithelial umbilical cord derived mesenchymal stem cells possess markers selected from a group comprising of; a) CD29; b) CD73; c) CD90; d) CD166; e) SSEA4; f) CD9; g) CD44; h) CD146; and i) CD105

Preferred methods include embodiments wherein said subepithelial umbilical cord derived mesenchymal stem cells do not express markers selected from a group comprising of; a) CD45; b) CD34; c) CD14; d) CD79; e) CD106; f) CD86; g) CD80; h) CD19; i) CD117; j) Stro-1 and k) HLA-DR.

Preferred methods include embodiments wherein said subepithelial umbilical cord derived mesenchymal stem cells express CD29, CD73, CD90, CD166, SSEA4, CD9, CD44, CD146, and CD105.

Preferred methods include embodiments wherein said subepithelial umbilical cord derived mesenchymal stem cells do not express CD45, CD34, CD14, CD79, CD106, CD86, CD80, CD19, CD117, Stro-1, and HLA-DR.

Preferred methods include embodiments wherein said subepithelial umbilical cord derived mesenchymal stem cells are positive for SOX2.

Preferred methods include embodiments wherein said subepithelial umbilical cord derived mesenchymal stem cells are positive for OCT4.

Preferred methods include embodiments wherein said subepithelial umbilical cord derived mesenchymal stem cells are positive for OCT4 and SOX2.

Preferred methods include embodiments wherein said immune cells are T cells.

Preferred methods include embodiments wherein said T cells are CD4 cells.

Preferred methods include embodiments wherein said T cells are CD8 cells.

Preferred methods include embodiments wherein said T cells are NKT cells.

Preferred methods include embodiments wherein said T cells are gamma delta T cells.

Preferred methods include embodiments wherein said T cells are Th1 cells.

Preferred methods include embodiments wherein said Th1 cells have a propensity for producing interferon gamma over interleukin 4 upon stimulation via the CD3 protein.

Preferred methods include embodiments wherein said Th1 cells express STAT4.

Preferred methods include embodiments wherein said Th1 cells express STAT1.

Preferred methods include embodiments wherein said Th1 cells express T-bet.

Preferred methods include embodiments wherein said Th1 cells express CCR1.

Preferred methods include embodiments wherein said Th1 cells express CCR5.

Preferred methods include embodiments wherein said Th1 cells express CXCR3.

Preferred methods include embodiments wherein said Th1 cells express CD119.

Preferred methods include embodiments wherein said Th1 cells express interferon gamma receptor II.

Preferred methods include embodiments wherein said Th1 cells express IL-18 receptor.

Preferred methods include embodiments wherein said Th1 cells express IL-12 receptor.

Preferred methods include embodiments wherein said Th1 cells express IL-27 receptor.

Preferred methods include embodiments wherein said T cells are Th2 cells.

Preferred methods include embodiments wherein said Th2 cells have a proclivity to produce more interleukin-4 than interferon gamma upon stimulation via CD3.

Preferred methods include embodiments wherein said Th2 cells express GATA-3.

Preferred methods include embodiments wherein said Th2 cells express IRF-4.

Preferred methods include embodiments wherein said Th2 cells express STAT5.

Preferred methods include embodiments wherein said Th2 cells express STAT6.

Preferred methods include embodiments wherein said Th2 cells express CCR3.

Preferred methods include embodiments wherein said Th2 cells express CCR4.

Preferred methods include embodiments wherein said Th2 cells express CCR8.

Preferred methods include embodiments wherein said Th2 cells express CXCR4.

Preferred methods include embodiments wherein said Th2 cells express interleukin-4 receptor.

Preferred methods include embodiments wherein said Th2 cells express interleukin-33 receptor.

Preferred methods include embodiments wherein said T cells are Th9 cells.

Preferred methods include embodiments, wherein said Th9 cell produces interleukin-9.

Preferred methods include embodiments wherein said Th9 cell expresses IRF4.

Preferred methods include embodiments wherein said Th9 cell expresses PU.1.

Preferred methods include embodiments wherein said Th9 cell secretes CCL17.

Preferred methods include embodiments wherein said Th9 cell secretes CCL22.

Preferred methods include embodiments wherein said Th9 cell secretes IL-10.

Preferred methods include embodiments wherein said Th9 cell expresses TGF-beta receptor II.

Preferred methods include embodiments wherein said T cell is a follicular helper T cell.

Preferred methods include embodiments wherein said follicular helper T cell expresses bcl-6.

Preferred methods include embodiments wherein said follicular helper T cell expresses c-maf.

Preferred methods include embodiments wherein said follicular helper T cell expresses stat-3.

Preferred methods include embodiments wherein said follicular helper T cell secretes CXCL-13.

Preferred methods include embodiments wherein said follicular helper T cell secretes interferon gamma.

Preferred methods include embodiments wherein said follicular helper T cell secretes interleukin-4.

Preferred methods include embodiments wherein said follicular helper T cell secretes IL-10.

Preferred methods include embodiments wherein said follicular helper T cell secretes IL-17A.

Preferred methods include embodiments wherein said follicular helper T cell secretes IL-17F.

Preferred methods include embodiments wherein said follicular helper T cell secretes IL-21.

Preferred methods include embodiments wherein said follicular helper T cell expresses BTLA-4.

Preferred methods include embodiments wherein said follicular helper T cell secretes CD40 ligand.

Preferred methods include embodiments wherein said follicular helper T cell expresses CD57.

Preferred methods include embodiments wherein said follicular helper T cell expresses CD84.

Preferred methods include embodiments wherein said follicular helper T cell expresses CXCR-4.

Preferred methods include embodiments wherein said follicular helper T cell expresses CXCR-5.

Preferred methods include embodiments wherein said follicular helper T cell expresses ICOS.

Preferred methods include embodiments wherein said follicular helper T cell expresses IL-6 receptor.

Preferred methods include embodiments wherein said follicular helper T cell expresses IL-21 receptor.

Preferred methods include embodiments wherein said follicular helper T cell expresses CD10.

Preferred methods include embodiments wherein said follicular helper T cell expresses OX40.

Preferred methods include embodiments wherein said follicular helper T cell expresses PD-1.

Preferred methods include embodiments wherein said follicular helper T cell expresses CD150.

Preferred methods include embodiments wherein said T cell is a Th17 cell.

Preferred methods include embodiments wherein said Th17 cell secretes interleukin-17A.

Preferred methods include embodiments wherein said Th17 cell secretes interleukin-17F.

Preferred methods include embodiments wherein said Th17 cell secretes IL-21.

Preferred methods include embodiments wherein said Th17 cell secretes IL-26.

Preferred methods include embodiments wherein said Th17 cell secretes CCL20.

Preferred methods include embodiments wherein said Th17 cell expresses BATF.

Preferred methods include embodiments wherein said Th17 cell expresses IRF4.

Preferred methods include embodiments wherein said Th17 cell expresses ROR alpha.

Preferred methods include embodiments wherein said Th17 cell expresses ROR gamma.

Preferred methods include embodiments wherein said Th17 cell expresses STAT3.

Preferred methods include embodiments wherein said Th17 cell expresses CCR4.

Preferred methods include embodiments wherein said Th17 cell expresses CCR6.

Preferred methods include embodiments wherein said Th17 cell expresses IL-1 receptor.

Preferred methods include embodiments wherein said Th17 cell expresses IL-6 receptor alpha.

Preferred methods include embodiments wherein said Th17 cell expresses IL-21 receptor.

Preferred methods include embodiments wherein said Th17 cell expresses IL-23 receptor.

Preferred methods include embodiments wherein said T cell is a Th22 cell.

Preferred methods include embodiments wherein said Th22 cell secretes IL-10.

Preferred methods include embodiments wherein said Th22 cell secretes IL-13.

Preferred methods include embodiments wherein said Th22 cell secretes FGF-1.

Preferred methods include embodiments wherein said Th22 cell secretes FGF-2.

Preferred methods include embodiments wherein said Th22 cell secretes FGF-5.

Preferred methods include embodiments wherein said Th22 cell secretes IL-21.

Preferred methods include embodiments wherein said Th22 cell secretes IL-22.

Preferred methods include embodiments wherein said Th22 cell expresses AHR.

Preferred methods include embodiments wherein said Th22 cell expresses batf.

Preferred methods include embodiments wherein said Th22 cell expresses STAT-3

Preferred methods include embodiments wherein said Th22 cell expresses CCR4.

Preferred methods include embodiments wherein said Th22 cell expresses CCR6.

Preferred methods include embodiments wherein said Th22 cell expresses CCR10.

Preferred methods include embodiments wherein said Th22 cell expresses IL-6 receptor.

Preferred methods include embodiments wherein said Th22 cell expresses TGF-beta receptor II.

Preferred methods include embodiments wherein said Th22 cell expresses TNF receptor 1.

Preferred methods include embodiments wherein said T cell is a T regulatory cell.

Preferred methods include embodiments wherein said T regulatory cell expresses foxp-3.

Preferred methods include embodiments wherein said T regulatory cell expresses Helios.

Preferred methods include embodiments wherein said T regulatory cell expresses STAT5.

Preferred methods include embodiments wherein said T regulatory cell expresses CD5.

Preferred methods include embodiments wherein said T regulatory cell expresses CD25.

Preferred methods include embodiments wherein said T regulatory cell expresses CD39.

Preferred methods include embodiments wherein said T regulatory cell expresses CD105.

Preferred methods include embodiments wherein said T regulatory cell expresses IL-7 receptor.

Preferred methods include embodiments wherein said T regulatory cell expresses CTLA-4.

Preferred methods include embodiments wherein said T regulatory cell expresses folate receptor.

Preferred methods include embodiments wherein said T regulatory cell expresses CD223

Preferred methods include embodiments wherein said T regulatory cell expresses LAP.

Preferred methods include embodiments wherein said T regulatory cell expresses GARP.

Preferred methods include embodiments wherein said T regulatory cell expresses Neuropilin.

Preferred methods include embodiments wherein said T regulatory cell expresses CD134.

Preferred methods include embodiments wherein said T regulatory cell expresses CD62 ligand.

Preferred methods include embodiments wherein said T regulatory cell secretes IL-10.

Preferred methods include embodiments wherein said T regulatory cell secretes TGF-alpha.

Preferred methods include embodiments wherein said T regulatory cell secretes TGF-beta.

Preferred methods include embodiments wherein said T regulatory cell secretes soluble TNF receptor p55.

Preferred methods include embodiments wherein said T regulatory cell secretes soluble TNF receptor p75.

Preferred methods include embodiments wherein said T regulatory cell secretes IL-2.

Preferred methods include embodiments wherein said T regulatory cell secretes soluble HLA-G.

Preferred methods include embodiments wherein said T regulatory cell secretes soluble Fas ligand.

Preferred methods include embodiments wherein said T regulatory cell secretes IL-35.

Preferred methods include embodiments wherein said T regulatory cell secretes VEGF.

Preferred methods include embodiments wherein said T regulatory cell secretes HGF.

Preferred methods include embodiments wherein said T regulatory cell secretes FGF1.

Preferred methods include embodiments wherein said T regulatory cell secretes FGF2.

Preferred methods include embodiments wherein said T regulatory cell secretes FGF5.

Preferred methods include embodiments wherein said T regulatory cell secretes Galectin 1.

Preferred methods include embodiments wherein said T regulatory cell secretes Galectin 9.

Preferred methods include embodiments wherein said T regulatory cell secretes IL-20.

Preferred methods include embodiments wherein said T regulatory cell expresses perforin.

Preferred methods include embodiments wherein said T regulatory cell expresses granzyme.

Preferred methods include embodiments wherein said T regulatory cell inhibits activation of a conventional T cell.

Preferred methods include embodiments wherein said conventional T cell does not express CD25.

Preferred methods include embodiments wherein said conventional T cell is activated by ligation of CD3.

Preferred methods include embodiments wherein said T conventional T cell is activated by ligation of CD3 and CD28.

Preferred methods include embodiments wherein said activation of said conventional T cell is proliferation.

Preferred methods include embodiments wherein said activation of said conventional T cell is cytokine secretion.

Preferred methods include embodiments wherein said cytokine is IL-2.

Preferred methods include embodiments wherein said cytokine is IL-4.

Preferred methods include embodiments wherein said cytokine is IL-6.

Preferred methods include embodiments wherein said cytokine is IL-10.

Preferred methods include embodiments wherein said cytokine is IL-12.

Preferred methods include embodiments wherein said cytokine is IL-7.

Preferred methods include embodiments wherein said cytokine is IL-13.

Preferred methods include embodiments wherein said cytokine is IL-15.

Preferred methods include embodiments wherein said cytokine is IL-18.

Preferred methods include embodiments wherein said immune cells are peripheral blood mononuclear cells.

Preferred methods include embodiments wherein said peripheral blood mononuclear cells are treated with an immune modulatory agent prior to contacting with said regenerative cells.

Preferred methods include embodiments wherein said immune modulatory agent is ultraviolet light.

Preferred methods include embodiments wherein said immune modulatory agent is HGF.

Preferred methods include embodiments wherein said immune modulatory agent is oxytocin.

Preferred methods include embodiments wherein said immune modulatory agent is NGF.

Preferred methods include embodiments wherein said immune modulatory agent is FGF-1.

Preferred methods include embodiments wherein said immune modulatory agent is FGF-2.

Preferred methods include embodiments wherein said immune modulatory agent is a toll like receptor activator.

Preferred methods include embodiments wherein oxytocin is administered to peripheral blood mononuclear cells in vitro for a period of 1 minute to 4 weeks.

Preferred methods include embodiments wherein oxytocin is administered to peripheral blood mononuclear cells in vitro for a period of 2 hours to 1 week.

Preferred methods include embodiments wherein oxytocin is administered to said fibroblasts in vitro for a period of 24 hours to 72 hours.

Preferred methods include embodiments wherein said oxytocin is administered at a concentration of 10 nM-10 uM.

Preferred methods include embodiments wherein said oxytocin is administered at a concentration of 100 nM-1 uM.

Preferred methods include embodiments wherein an immune modulatory agent is administered to the combination of immune cells and regenerative cells.

Preferred methods include embodiments wherein said immune modulatory agent is curcumin.

Preferred methods include embodiments wherein said immune modulatory agent is TGF-beta.

Preferred methods include embodiments wherein said immune modulatory agent is galectin-1.

Preferred methods include embodiments wherein said immune modulatory agent is galectin-3.

Preferred methods include embodiments wherein said immune modulatory agent is galectin-9.

Preferred methods include embodiments wherein said immune modulatory agent is IL-1.

Preferred methods include embodiments wherein said immune modulatory agent is IL-2.

Preferred methods include embodiments wherein said immune modulatory agent is IL-4.

Preferred methods include embodiments wherein said immune modulatory agent is IL-7.

Preferred methods include embodiments wherein said immune modulatory agent is IL-10.

Preferred methods include embodiments wherein said immune modulatory agent is IL-13.

Preferred methods include embodiments wherein said immune modulatory agent is IL-15.

Preferred methods include embodiments wherein said immune modulatory agent is IL-12.

Preferred methods include embodiments wherein said immune modulatory agent is IL-18.

Preferred methods include embodiments wherein said immune modulatory agent is IL-20.

Preferred methods include embodiments wherein said immune modulatory agent is IL-22.

Preferred methods include embodiments wherein said immune modulatory agent is IL-35.

### BRIEF DESCRIPTION OF THE DRAWINGS

FIG. 1 is a bar graph showing the effects of reprogrammed immune cells on ALT levels.

### DESCRIPTION OF THE INVENTION

For the practice of the invention, MSC are used to reprogram immune cells in order to endow liver regenerative activity can be utilized as was previously performed in clinical trials with non-selected MSC. “Mesenchymal stem cell” or “MSC” in some embodiments refers to cells that are (1) adherent to plastic, (2) express CD73, CD90, and CD105 antigens, while being CD14, CD34, CD45, and HLA-DR negative, and (3) possess ability to differentiate to osteogenic, chondrogenic and adipogenic lineage. Other cells possessing mesenchymal-like properties are included within the definition of “mesenchymal stem cell”, with the condition that said cells possess at least one of the following: a) regenerative activity; b) production of growth factors; c) ability to induce a healing response, either directly, or through elicitation of endogenous host repair mechanisms. As used herein, “mesenchymal stromal cell” or ore mesenchymal stem cell can be used interchangeably. Said MSC can be derived from any tissue including, but not limited to, bone marrow, adipose tissue, amniotic fluid, endometrium, trophoblast-derived tissues, cord blood, Wharton jelly, placenta, amniotic tissue, derived from pluripotent stem cells, and tooth. In some definitions of “MSC”, said cells include cells that are CD34 positive upon initial isolation from tissue but are similar to cells described about phenotypically and functionally. As used herein, “MSC” may includes cells that are isolated from tissues using cell surface markers selected from the list comprised of NGF-R, PDGF-R, EGF-R, IGF-R, CD29, CD49a, CD56, CD63, CD73, CD105, CD106, CD140b, CD146, CD271, MSCA-1, SSEA4, STRO-1 and STRO-3 or any combination thereof, and satisfy the ISCT criteria either before or after expansion. Furthermore, as used herein, in some contexts, “MSC” includes cells described in the literature as bone marrow stromal stem cells (BMSSC), marrow-isolated adult multipotent inducible cells (MIAMI) cells, multipotent adult progenitor cells (MAPC), mesenchymal adult stem cells (MASCS), MultiStem®, Prochymal®, remestemcel-L, Mesenchymal Precursor Cells (MPCs), Dental Pulp Stem Cells (DPSCs), PLX cells, PLX-PAD, AlloStem®, Astrostem®, Ixmyelocel-T, MSC-NTF, NurOwn™, Stemedyne™-MSC, Stempeucel®, StempeucelCLI, StempeucelOA, HiQCell, Hearticellgram-AMI, Revascor®, Cardiorel®, Cartistem®, Pneumostem®, Promostem®, Homeo-GH, AC607, PDA001, SB623, CX601, AC607, Endometrial Regenerative Cells (ERC), adipose-derived stem and regenerative cells (ADRCs).

Mesenchymal stem cells (MSCs) are adult stem cells with self-renewing abilities[40] and have been shown to differentiate into a wide range of tissues including mesoderm- and nonmesoderm-derived[40, 41], such as hepatocytes[42-47]. MSCs are capable of entering and maintaining satellite cell niches, particularly in hematopoiesis[48, 49], and are key in tissue repair and regeneration, aging, and regulating homeostasis[50-53]. In the case of liver failure, MSCs can aid in regeneration of hepatic tissue[54-60], and their interactions with the immune system[61-67] have potential as adjuvants during organ transplants[68], including liver transplantation[69].

MSCs were discovered in 1970 by Friedenstein et al[70] who demonstrated that bone marrow (BM) contained both hematopoietic stem cells (HSCs), which are non-plastic adherent, and a population of a more rare adherent cell. The adherent cells were able to form single cell colonies and were referred to as stromal cells. Those stromal cells, which are capable of self-renewal and expansion in culture are now referred to as mesenchymal stem cells (MSCs). Friedenstein was the first to show that MSCs could differentiate into mesoderm and to demonstrate their importance in controlling the hematopoietic niche [71].

In the 1980s, more research on MSCs found that they could differentiate into muscle, cartilage, bone and adipose-derived cells [72]. Caplan et al showed that MSCs are responsible for bone and cartilage regeneration induced by local cuing and genetic potential[73].

In the 1990s, Pittenger et al isolated MSCs from bone marrow and found that they retained their multilineage potential after expanding into selectively differentiated adipocytic, chondrocytic, or osteocytic lineages [41]. Likewise, Kopen et al showed that bone marrow MSCs differentiated into neural cells when exposed to the brain microenvironment[74]. In 1999, Petersen et al found that bone marrow-derived stem cells could be a source of hepatic oval cells in a rat model[36]. Specifically, they used male to female bone marrow transplant and subsequently induced blockade of hepatocyte proliferation by administration of a hepatotoxin followed by partial hepatectomy. As previously described, this procedure stimulates proliferation of LPC or “reserve cells” which generate new hepatocytes, such cells having previously identified as oval cells. Subsequent to the hepatectomy, Y chromosome, dipeptidyl peptidase IV enzyme, and L21-6 antigen were used to identify the newly generated oval cells, and their hepatocytic progeny to be of bone marrow origin.

The first decade of the 21^{st} century saw a surge of research on MSCs, leading to a greater understanding of their nature and of the cellular process behind regeneration [40, 52, 53]. In 2005, Teratani et al identified growth factors allowing hepatic fate-specification in mice and showed that embryonic stem cells could differentiate into functional hepatocytes [75]. In 2007, Chamberlain et al generated human hepatocytes from clonal MSCs in fetal sheep hepatic tissue, differentiating into hepatocytes both throughout the liver parenchyma and the periportal space [76]. The attractive features of MSC for clinical development include their ease of expansion, lack of need for donor matching, and standardized protocols for manufacturing and administration. Of particular interest for liver conditions is the observation that intravenous administration of MSC results in a primary homing of cells to the lung, followed by homing and retention to liver [77]. A unique property of MSC is their apparent hypoimmunogenicity and immune modulatory activity [78], which is present in MSC derived from various sources [79]. This is believed to account for the ability to achieve therapeutic effects in an allogeneic manner. Allogeneic bone marrow derived MSC have been used by academic investigators with clinical benefit treatment of diseases such as graft versus host (GVHD) [80-85], osteogenesis imperfecta [86], Hurler syndrome, metachromatic leukodystrophy [87], and acceleration of hematopoietic stem cell engraftment [88-90]. The company Athersys has successfully completed Phase I safety studies using allogeneic bone marrow MSCs is now in efficacy finding clinical trials (Phase II and Phase III) for Multiple Sclerosis, Crohn's Disease, and Graft Versus Host Disease using allogeneic bone marrow derived MSC. Intravenous administration of allogeneic MSCs by Osiris was also reported to induce a statistically significant improvement in cardiac function in a double-blind study [91].

Currently there several MSC-based therapies that have received governmental approvals including Prochymal™ which was registered in Canada and New Zealand for treatment of graft versus host disease [92, 93]. Although in terms of clinical translation bone marrow MSC are the most advanced, several other sources of MSC are known which possess various properties that may be useful for specific conditions. Bone marrow is also a source for hematopoietic stem cells (HSCs), which have also been used for liver regeneration. Likewise, human placenta is an easily accessible source of abundant MSCs, which can be differentiated in vitro. Finally, MSCs with tissue regenerative abilities can also be isolated from adipose tissue and induced to hepatocytes in large numbers.

In some embodiments of the invention reprogrammed immune cells are administered with MSC for treatment of liver failure. The discussion below provides examples of the use of MSC in liver failure, which may be useful for one of skill in the art to combine MSC with reprogrammed immune cells

Early studies suggested that out of the hepatic regenerative cells found in the bone marrow that the MSC component is the most regenerative cell type as compared to other cell types such as hematopoietic stem cells [44]. Given the fact that BM-MSC are capable of differentiating into various tissues in vitro, combined with the putative bone marrow origin of the hepatic-repairing oval cell [36], investigators sought to determine whether BM-MSC could be induced to differentiate into hepatocyte cells in vitro through culture in conditions that would imitate hepatic regeneration. Lee et al developed a 2-step protocol for hepatocyte differentiation using culture in hepatocyte growth factor, followed by oncostatin M. After 4 weeks of induction the investigators reported the spindle-like BM-MSC taking a cuboidal morphology, which is characteristic of hepatocytes. Furthermore the differentiating cells were seen to initiate expression of hepatic-specific genes in a time-dependent manner correlating with morphological changes. From a functional perspective, the generated hepatocytes exhibited features of liver cells, specifically albumin production, glycogen storage, urea secretion, uptake of low-density lipoprotein, and phenobarbital-inducible cytochrome P450 activity [43]. To improve yield and potency of BM-MSC generated hepatocytes, Chen et al utilized conditioned media from cultured hepatocytes as part of the differentiation culture conditions. They reported that BM-MSC cultures in the differentiation conditions started taking an epithelioid, binucleated morphology at days 10 and 20. Gene assessment revealed increase in AFP, HNF-3beta, CK19, CK18, ALB, TAT, and G-6-Pase mRNA, which was confirmed at the protein levels. Additionally, the cells started taking a functional phenotype similar to hepatocytes, including, hepatocyte-like cells by culture in conditioned medium further demonstrated in vitro functions characteristic of liver cells, including glycogen storage, and urea secretion activities. In vivo relevance of these artificially generated hepatic like cells was seen in that restoration of albumin activity and suppression of liver enzymes was seen upon transplantation to immune deficient animals exposed to chemically induced liver injury [94]. In accordance with the concept that injured tissue mediates MSC activation and subsequent repair, Mohsin et al demonstrated that coculture of BM-MSC with chemically-injured hepatocytes augments hepatic differentiation as compared to coculture with naïve hepatocytes [95].

Based on in vitro differentiation, as well as the possibility of MSC producing cytokines such as HGF [96-98], which are known to stimulate hepatic regeneration/decrease hepatocyte apoptosis [99-106], several animal studies were conducted using BM-MSC in models of liver injury. Fang et al utilized a carbon tetrachloride induced hepatic injury model to assess effects of systemically administered BM-MSC on fibrosis and hepatocyte demise. It was found that MSC infusion after exposure to the hepatotoxin significantly reduced liver damage and collagen deposition. Supporting the possibility of a concurrent protective and regenerative effect, levels of hepatic hydroxyproline and serum fibrosis markers in mice receiving cells were significantly lower compared with those of control mice. Histologic examination suggested that hepatic damage recovery was accelerated in the treated mice. Donor cell engraftment and possible in vivo hepatic differentiation was supported by immunofluorescence, polymerase chain reaction, and fluorescence in situ hybridization analysis, which demonstrated donor-derived cells possessing epithelium-like morphology expressed albumin [107]. Interestingly, the amount of engrafted cells was minute and could not explain the functional recovery of serum albumin, suggesting the possibility of paracrine effects. A subsequent study using the same carbon tetrachloride model demonstrated that BM-MSC administration resulted to reactive oxygen species ex vivo, reduced oxidative stress in recipient mice, and accelerated repopulation of hepatocytes after liver damage [56]. To optimize the route of administration, Zhao et al assessed intravenous, intrahepatic, and intraperitoneal administration of BM-MSC in rats treated with carbon tetrachloride. Functional recovery was most profound in the intravenous administration group, which was correlated with increased IL-10 and decreased IL-1, TNF-alpha, and TGF-beta. Furthermore, in vivo differentiation of the BM-MSC was observed based on expression of α-fetoprotein, albumin, and cytokeratin 18 in cells deriving from donor origin [108].

In order to assess whether the therapeutic effects of BM-MSC are specific to the carbon tetrachloride model, or whether they may be extrapolated to other models of hepatic injury, investigators assessed whether BM-MSC are useful in hepatectomy recovery models. While recovery is generally observed after ⅔ or 70% hepatectomy, 90% hepatectomy is lethal in rats. In one study, BM-MSC were differentiated in vitro by culture on Matrigel with hepatocyte growth factor and fibroblast growth factor-4 into cells expressing hepatocyte-like properties. Specifically, the cells expressed a hepatic-like cuboidal morphology and were positive for albumin, cytochrome P450 (CYP) 1A1, CYP1A2, glucose 6-phosphatase, tryptophane-2,3-dioxygenase, tyrosine aminotransferase, hepatocyte nuclear factor (HNF)1 alpha, and HNF4alpha. Intrasplenic administration of differentiated cells subsequent to the 90% hepatectomy resulted prevention of lethality [109]. Another study confirmed efficacy of BM-MSC at accelerating post-hepatectomy liver regeneration subsequent to intraportal administration. Regenerative effects where associated with upregulation of HGF expression in the newly synthesized tissue [110]. It is interesting that the regenerative effects of BM-MSC are observed not only in acute settings but also in chronic conditions leading to liver failure. Non-alcoholic steatohepatitis (NASH) is a precursor to cirrhosis and is characterized by lipid accumulation, hepatocyte damage, leukocyte infiltration, and fibrosis. It was demonstrated that in C57BL/6 mice chronically fed with high-fat diet, that the intravenous administration of BM-MSC resulted in reduction of plasma levels of hepatic enzyme, hepatomegaly, liver fibrosis, inflammatory cell infiltration, and inflammatory cytokine gene expression, as compared to control mice [111]. Overall, these data suggest that BM-MSC has some repairative/regenerative activity on livers that are damaged in either chronic or acute settings.

Additional animal studies were conducted in both chronic and acute liver toxicity settings. For example, Hwang et al [112], treated Sprague-Dawley rats with 0.04% thioacetamide (TAA)-containing water for 8 weeks, and BM-MSC were injected into the spleen with the intent of transsplenic migration into the liver. Ingestion of TAA for 8 weeks induced micronodular liver cirrhosis in 93% of rats. Examination of MSC microscopically revealed that the injected cells were diffusely engrafted in the liver parenchyma, differentiated into CK19 (cytokeratin 19)- and thy1-positive oval cells and later into albumin-producing hepatocyte-like cells. MSC engraftment rate per slice was measured as 1.0-1.6%. MSC injection resulted in apoptosis of hepatic stellate cells and resultant resolution of fibrosis, but did not cause apoptosis of hepatocytes. Given that stellate cells are responsible for matrix deposition and fibrosis [113, 114], this is an interesting observation. Injection of MSCs treated with HGF in vitro for 2 weeks, which became CD90-negative and CK18-positive, resulted in chronological advancement of hepatogenic cellular differentiation by 2 weeks and decrease in anti-fibrotic activity. Mechanistically, it appeared that the BM-MSC directly differentiated to oval cells and hepatocytes, which was associated with repair of damaged hepatocytes, intracellular glycogen restoration and resolution of fibrosis.

An acute model of liver failure is produced by administration to animals of D-galactosamine, a TNF-alpha stimulating hepatotoxin [115], and lipopolysaccharide (LPS) a potent inflammatory stimulus that replicates translocation of gut bacteria often seen in liver failure [116, 117]. In this model it was demonstrated that administration of BM-MSC in pretreated rats resulted in reduction of ALT, AST, caspase-1 and IL-18 proteins, and mRNA as compared to the control group [118]. Mechanistic elucidation at a cellular level demonstrated that the injected BM-MSC were inhibiting hepatocyte apoptosis. Interestingly the authors also found that recovering animals possessed higher levels of VEGF protein as compared to non-treated animals. This is intuitively logical given that VEGF is a key cytokine in the angiogenesis cascade, and angiogenesis seems to be required regression of liver failure [119-122]. Using the same D-galactosamin/LPS model, Sun et al [123], sought to identify optimal route of delivery for BM-MSC. They divided rats into the following groups: a) hepatic artery injection; b) portal vein injection; c) tail vein injection group; and d) intraperitoneal injection. They found that compared with the control group, ALT, AST, and damage to the liver tissue in the hepatic artery group, the portal vein group and the tail vein group improved in vivo. The expression of PCNA and HGF in the liver was higher and caspase-3 expression was lower in the hepatic artery injection group, the portal vein injection group and the tail vein injection group than that in the intraperitoneal injection and control groups. The BRdU-labeled BM-MSCs were only observed homing to the liver tissue in these three groups. However, no significant differences were observed between these three groups. Liver function was improved following BM-MSC transplantation via 3 endovascular implantation methods (through the hepatic artery, portal vein and vena caudalis). These data suggest that intra-hepatic artery injection was most effective and that intraperitoneal administration is ineffective. A large animal study using similar hepatotoxins was performed in the pig. Li et al. administered 3×10(7) human BM-MSC via the intraportal route or peripheral vein immediately after D-galactosamine injection, and a sham group underwent intraportal transplantation (IPT) without cells (IPT, peripheral vein transplantation [PVT], and control groups, respectively, n=15 per group). All of the animals in the PVT and control groups died of FHF within 96 hours. In contrast, 13 of 15 animals in the IPT group achieved long-term survival (>6 months). Immunohistochemistry demonstrated that transplanted human BM-MSC-derived hepatocytes in surviving animals were widely distributed in the hepatic lobules and the liver parenchyma from weeks 2 to 10. Thirty percent of the hepatocytes were BM-MSC-derived. However, the number of transplanted cells decreased significantly at week 15. Only a few single cells were scattered in the regenerated liver lobules at week 20, and the liver tissues exhibited a nearly normal structure. These data suggest that intraportal delivery may be ideal and also reinforce the notion that MSC may be transplanted across allo and xeno barriers without need for immune suppression.

Clinical trials utilizing BM-MSC have shown an excellent safety profile, with various levels of efficacy in liver failure. Mohamadnejad et al [59], conducted a 4 patient study with decompensated liver cirrhosis. Patoemt bone marrow was aspirated, mesenchymal stem cells were cultured, and a mean 31.73×10(6) mesenchymal stem cells were infused through a peripheral vein. There were no side-effects in the patients during follow-up. The model for end-stage liver disease (MELD) scores of patients 1, and 4 improved by four and three points, respectively by the end of follow-up. Furthermore, the quality of life of all four patients improved by the end of follow-up. Using SF-36 questionnaire, the mean physical component scale increased from 31.44 to 65.19, and the mean mental component scale increased from 36.32 to 65.55. Another study treated 8 patients (four hepatitis B, one hepatitis C, one alcoholic, and two cryptogenic) with end-stage liver disease having MELD score > or =10 were included. Autologous BM-MSCs were taken from iliac crest. Approximately, 30-50 million BM-MSCs were proliferated and injected into peripheral or the portal vein. Subsequent to experiment the MELD Score was decreased from 17.9+/−5.6 to 10.7+/−6.3 (P<0.05) and prothrombin complex from international normalized ratio 1.9+/−0.4 to 1.4+/−0.5 (P<0.05). Serum creatinine decreased from 114+/−35 to 80+/−18 micromol/l (P<0.05). This trial supports the safety with signal of efficacy of the BM-MSC activity in liver failure clinically.

A larger trial of autologous BM-MSC focused on patients with liver failure associated with hepatitis B infection [124]. Part of the rational was previous studies showing that BM-MSC derived hepatocytes are resistant to hepatitis B infection [125]. Peng et al [124], treated 53 patients and as controls used 105 patients matched for age, sex, and biochemical indexes, including alanine aminotransferase (ALT), albumin, total bilirubin (TBIL), prothrombin time (PT), and MELD score. In the 2-3 week period after cell administration, efficacy was observed based on levels of ALB, TBIL, and PT and MELD score, compared with those in the control group. Safety of the procedure was demonstrated in that there were no differences in incidence of hepatocellular carcinoma (HCC) or mortality between the treated and control groups at 192 weeks. Unfortunately, liver function between the two groups was also similar at 192 weeks, suggesting the beneficial effects of BM-MSC were transient in nature. Supporting the possibility of transient effects of BM-MSC was a 27 patient study in patients with decompensated cirrhosis in which 15 patients received BM-MSC and 12 patients received placebo. The absolute changes in Child scores, MELD scores, serum albumin, INR, serum transaminases and liver volumes did not differ significantly between the MSC and placebo groups at 12 months of follow-up. Unfortunately the publication did not provide 3 or 6 month values [126]. In contrast, a more recent study administered BM-MSC into 12 patients (11 males, 1 female) with baseline biopsy-proven alcoholic cirrhosis who had been alcohol free for at least 6 months [127]. A 3 month assessment histological improvement and reduction of fibrosis was quantified according to the Laennec fibrosis scoring scale in 6 of 11 patients. Additionally, at 3 months post cell administration, the Child-Pugh score improved significantly in ten patients and the levels of transforming growth factor-01, type 1 collagen and α-smooth muscle actin significantly decreased (as assessed by real-time reverse transcriptase polymerase chain reaction) after BM-MSCs therapy. Overall the different underlying conditions, route of administration, and time points of assessments between studies makes it difficult to draw solid conclusions, although it appears that some therapeutic effect exists, although longevity of effect is not known.

Given that one possibility for the lack of efficacy long term in the previous study may be inappropriate level of hepatocyte differentiation in vivo, Amer et al conducted a clinical trial where BM-MSC were pre-differentiated toward the hepatocyte lineage by a culture cocktail containing HGF [128]. They conducted a 40 patient trial in hepatitis C patients in which 20 patients were treated with partially differentiated cells either intrasplenically or intrahepatically and 20 patients received placebo control. At the 3 and 6 month time points a significant improvement in ascites, lower limb edema, and serum albumin, over the control group was observed. Additionally significant benefit was quantified in the Child-Pugh and MELD scores. No difference was observed between intrahepatic or intrasplenic administration. This study demonstrates the potential of semi-differentiated hepatocytes from BM-MSC to yield therapeutic benefit without reported adverse effects.

Out of the BM-MSC studies described, one potential reason for relatively mediocre results could be the fact that autologous cells where utilized in all of the studies. While autologous MSC possess the benefit of lack of immunogenicity, a drawback may be a relative dysfunction of these cells given the poor health condition of the patients. Indeed several studies have demonstrated that MSC from patients suffering from chronic conditions possess inhibited regenerative activity when compared with MSC from healthy donors [129-135].

Adipose tissue is an attractive alternative to bone marrow as a source of stem cells for treatment of degenerative conditions in general and liver failure specifically [46, 136], for the following reasons: a) extraction of adipose derived cells is a simpler procedure that is much less invasive than bone marrow extraction; b) Adipose tissue contains a higher content of mesenchymal stem cells (MSC) as compared to bone marrow, therefore shorter in vitro expansion times are needed; and c) MSC from adipose tissue do not decrease in number with aging [137-139]. Adipose tissue derived MSC were originally described by Zuk et al who demonstrated the stromal vascular fraction (SVF) of adipose tissue contains large numbers of cells that could be induced to differentiate into adipogenic, chondrogenic, myogenic, and osteogenic lineages and morphologically resembled MSC [140]. Subsequent to the initial description, the same group reported that in vitro expanded SVF derived cells had surface marker expression similar to bone marrow derived MSC, displaying expression of CD29, CD44, CD71, CD90, CD105/SH2, and SH3 and lacking CD31, CD34, and CD45 expression [141]. This suggested that SVF expanded adherent cells where indeed members of the MSC family, a notion that has subsequently gained acceptance [142-144]. To date, clinical trials on adipose derived cells have all utilized ex vivo-expanded cells, which share properties with bone marrow derived MSC [145-150]. Preparations of MSC expanded from adipose tissue are equivalent or superior to bone marrow in terms of differentiation ability [151, 152], angiogenesis-stimulating potential [153], and immune modulatory effects [79, 154].

In the area of liver failure Banas et al created a 13 day in vitro differentiation protocol to generate hepatocyte like cells from human adipose tissue MSC (AT-MSC) [42]. The differentiated cells possessed a hepatocyte-like morphology and phenotypically resembled primary hepatocytes. Administration of the cells in a carbon tetrachloride induced liver failure model resulted in diminished liver injury, AST, ALT, as well as ammonia. Unfortunately comparison with BM-MSC was not performed. A subsequent study utilized AT-MSC that were not differentiated and injected into the tail vein [155]. Administration of cells led to death in 4 of 6 mice due to lung infarction, presumably as a result of cell accumulation in pulmonary microcapillaries. To overcome this the investigators utilized a combination of AT-MSC and heparin, this resulted in trend, which did not reach significance, for reduced ALT, AST, and LDH in the treated group. It was demonstrated in a subsequent tracking study by the same group that heparin decreased pulmonary retention and increased hepatic retention by 30% [156]. In order to elucidate whether alternative routes of AT-MSC administration may augment therapeutic activities, Kim et al [157], assessed intravenous, intrahepatic parenchyma, and intra-portal vein delivery of cells in the same carbon tetrachloride model as utilized by the previous two experiments. They found that all 3 routes led to significant decrease in histological injury as well as AST, ALT, and ammonia. The most profound protective effects where observed with the intravenous route was used. One possible reason for statistical significant efficacy in this study and not in the previous study may be that in this study AT-MSC were injected at day 1 and 3 after carbon tetrachloride administration, whereas the previous study involved only one injection. The previous AT-MSC experiments utilized human cells administered in animals, Deng et al [158], utilized syngeneic AT-MSC that were derived from mice transgenic with enhanced green fluorescent protein (eGFP) in mice treated with carbon tetrachloride. The survival rate of cell treated group significantly increased compared to PBS group. Furthermore, the transplanted cells were well integrated into injured livers and produced albumin, cytokeratin-18. Overall, it appears that in the carbon tetrachloride model both xenogeneic and syngenic AT-MSC have therapeutic effects, however standardization of protocols and models is needed to obtain a clearer picture of potency of effects.

Other models of hepatic injury have been utilized with AT-MSC. Salomone et al assessed human AT-MSC transfected with eGFP in rats treated with a hepatoxic dose of acetaminophen [159]. It was found that AT-MSC infusion decreased AST, ALT and prothrombin time to the levels observed in control rats. Furthermore clinical signs of liver failure such as encephalopathy were not observed in treated animals. Histologically, control animals displayed lobular necrosis and diffuse vacuolar degeneration, which was not seen in the treated group. Mechanistically, transplanted AT-MSC induced an increase in antioxidant status and decrease in inflammatory cytokines in the recipients. Additionally, proliferation of endogenous hepatocytes was observed. These data suggest that AT-MSC effects on liver injury are not limited to carbon tetrachloride but may be more widespread. Indeed another study utilized two chemicals that block hepatocyte regeneration together with partial hepatectomy. Specifically, using a model of a toxic liver damage in Sprague Dawley rats, generated by repetitive intraperitoneal application of retrorsine and allyl alcohol followed by two third partial hepatectomy, investigators assessed the regenerative effects of human AT-MSC. Six and twelve weeks after hepatectomy, animals were sacrificed and histological sections were analyzed. AT-MSC treated animals exhibited significantly raised albumin, total protein, glutamic oxaloacetic transaminase and LDH. The infused cells were found up to twelve weeks after surgery in histological sections. Although to our knowledge clinical studies of AT-MSC in liver disease have not been reported, one clinical trial (NCT01062750) is reported to be enrolling. This trial, run by Shuichi Kaneko of Kanazawa University in Japan comprises of intra-hepatic administration of AT-MSC.

Although numerous studies have examined the ability of MSC to induce hepatic regeneration, the original studies that demonstrated BM liver regenerative effects suggested that other cells in the BM compartment besides MSC may have therapeutic activities [160]. Given that bone marrow mononuclear cells (BMMC) have demonstrated therapeutic activities in numerous ischemic and chronic conditions [161-166], investigators sought to assess whether this mixture of cells would possess activity in animal models of liver failure. Terai et al administered BMMC isolated from mice transgenic for GFP to mice whose livers where injured by carbon tetrachloride. It was observed that the transplanted GFP-positive BMMC migrated into the peri-portal area of liver lobules after one day, and repopulated as much as 25% of the recipient liver by 4 weeks. Interestingly when mice where administered BMMC but not carbon tetrachloride, no donor cells could be detected at 4 weeks, indicating that injury must be present for long term hepatic retention. It appeared that the transplanted BMMC differentiated into functional mature hepatocytes which would overtake function of hepatocytes from carbon tetrachloride injured mice [167]. A subsequent study by the same group examined mechanisms of the antifibrotic/regenerative effect of the BMMC and found matrix metalloprotease (MMP) activation to be involved [168]. MMPs are important in liver regeneration not only because of their ability to cleave through fibrotic tissue in order to alter the local environment, but also because of their role in angiogenesis, which is important for liver regeneration [169-171]. Interestingly, regression of liver fibrosis by dendritic cells also is mediated through MMP activation [172].

One of the first clinical uses of BMMC in the liver involved purification of CD133 positive cells prior to administration, with the notion that CD133 selects for cells with enhanced regenerative potential [173]. Additionally, the CD133 subset of bone marrow cells may represent a hepatogenic precursor cell since cells of this phenotype are mobilized from the bone marrow subsequent to partial hepatectomy [174-176]. Another interesting point is that CD133 has been reported by some to be expressed on oval cells in the liver, although the bone marrow origin is controversial [177-179]. In 2005 am Esch et al described 3 patients subjected to intraportal administration of autologous CD133(+) BMSCs subsequent to portal venous embolization of right liver segments, used to expand left lateral hepatic segments. Computerized tomography scan volumetry revealed 2.5-fold increased mean proliferation rates of left lateral segments compared with a group of three consecutive patients treated without application of BMSCs [180]. In 2012 the same group reported on 11 patients treated with this procedure and 11 controls. They reported that mean hepatic growth of segments II/III 14 days after portal vein embolization in the group that received CD133 cells was significantly higher (138.66 mL±66.29) when compared with the control group (62.95 mL±40.03; P=0.004). Post hoc analysis revealed a better survival for the group that received cells as compared to the control. A similar study by another group involved 6 patients receiving CD133 cells to accelerate left lateral segment regeneration, with 7 matched control patients. The increase of the mean absolute future liver remnant volume (FLRV) in the treated group from 239.3 mL+/−103.5 to 417.1 mL+/−150.4 was significantly higher than that in the control group, which was from 286.3 mL+/−77.1 to 395.9 mL+/−94.1. The daily hepatic growth rate in the treated group (9.5 mL/d+/−4.3) was significantly higher to that in the control group (4.1 mL/d+/−1.9) (P=0.03). Furthermore, time to surgery was 27 days+/−11 in the treated group and 45 days+/−21 in the control group (P=0.057). These data suggest that in the clinical situation, CD133 cells isolated from BMMC appear to accelerate liver regeneration.

Another purified cell type from BMMC is CD34 expressing cells, which conventionally are known to possess the hematopoietic stem cell compartment [181]. Additionally, similar to CD133, CD34 is found on oval cells in the liver, suggesting possibility that bone marrow derived CD34 cells play a role in liver regeneration when hepatocyte proliferation is inhibited [182, 183]. Gordon et al [184], reported 5 patients with liver failure that were treated with isolated CD34 positive cells. Interestingly, instead of collected the cells from bone marrow harvest, the investigators mobilized the bone marrow cells by treatment with G-CSF. The investigators first demonstrated that these CD34 cells were capable of differentiating in vitro into albumin producing hepatocyte-like cells. A pilot clinical investigation was attempted in 5 patients with liver failure. The CD34 cells were injected into the portal vein (three patients) or hepatic artery (two patients). No complications or specific side effects related to the procedure were observed. Three of the five patients showed improvement in serum bilirubin and four of five in serum albumin. A subsequent publication by the same group reported the improvement in bilirubin levels was maintained for 18 months [185]. A subsequent case report by Gasbarrini et al [185]. described use of autologous CD34⁺ BMMC administered via the portal vein as a rescue treatment in an alcoholic patient with nimesulide-induced acute liver failure. A liver biopsy performed at 20 days following infusion showed augmentation of hepatocyte replication around necrotic foci; there was also improvement in synthetic liver function within the first 30 days.

Subsequent to the initial studies on CD133 and CD34 cells, investigators assessed the effects of unpurified BMMC on liver failure. Terai et al [60], treated 9 patients with liver cirrhosis from a variety of causes with autologous BMMC administered intravenously. Significant improvements in serum albumin levels and total protein were observed at 24 weeks after BMMC therapy. Significantly improved Child-Pugh scores were seen at 4 and 24 weeks. alpha-Fetoprotein and proliferating cell nuclear antigen (PCNA) expression in liver biopsy tissue was significantly elevated after BMMC infusion. No major adverse effects were noted. A subsequent study in alcohol associated decompensated liver failure examined effects of autologous BMMC administered intraportally in 28 patients compared to 30 patients receiving standard medical care. After 3 months, 2 and 4 patients died in the BMMC and control groups, respectively. Adverse events were equally distributed between groups. The MELD score improved in parallel in both groups during follow-up. Comparing liver biopsy at 4 weeks to baseline, steatosis improved, and proliferating HPC tended to decrease in both groups [186]. It is unclear why this larger study generated a negative outcome compared to the initial smaller study. Interestingly in another study in which 32 patients with decompensating liver cirrhosis were treated with autologous BMMC and 15 patients received standard of care, significant improvements were observed. Specifically, improvements in ALT, AST, albumin, bilirubin and histological score where observed. The efficacy of BMMC transplantation lasted 3-12 months as compared with the control group. Serious complications such as hepatic encephalopathy and spontaneous bacterial peritonitis were also significantly reduced in BM-MNCs transfused patients compared with the controls. However, these improvements disappeared in 24 months after transplantation [187]. It is possible that effects of BMMC are transient in liver failure, lasting less than 12 months. For example, Lyra et al [188], reported on 10 patients with Child-Pugh B and C liver failure who received autologous BMMC. Bilirubin levels were lower at 1 (2.19+/−0.9) and 4 months (2.10+/−1.0) after cell transplantation that baseline levels (2.78+/−1.2). Albumin levels 4 months after BMMC infusion (3.73+/−0.5) were higher than baseline levels (3.47+/−0.5). International normalized ratio (INR) decreased from 1.48 (SD=0.23) to 1.43 (SD=0.23) one month after cell transplantation. A larger study by the same group utilizing similar methodology reported similar transient benefit [189]. Specifically, a 30 patient study was conducted with hepatic cirrhosis patients on the transplant list who were randomized to receive BMMC or supportive care. Child-Pugh score improved in the first 90 days in the cell therapy group compared with controls. The MELD score remained stable in the treated group but increased during follow-up in the control group. Albumin levels improved in the treatment arm, whereas they remained stable among controls in the first 90 days. Bilirubin levels increased among controls, whereas they decreased in the therapy arm during the first 60 days; INR RC differences between groups reached up to 10%. The changes observed did not persist beyond 90 days.

Other means of utilizing bone marrow stem cells for hepatic regeneration include stimulating mobilization of endogenous stem cells by providing agents such as G-CSF. Experimental studies to investigate the mobilization of HSCs for hepatocyte formation have yielded conflicting results[190-192], but Shitzu et al in 2012 showed beneficial effects in a murine model of acute liver failure[193].

Several experimental studies have shown that MSCs isolated from human placenta promote healing in diseased rat livers, with an anti-fibrotic effect in liver cirrhosis[194] or reduction of fibrotic tissue[95]. By transplanting placenta-derived MSCs in the portal vein, Cao et al observed promising results in pigs, not only by producing hepatocytes but also by prolonging survival time, reducing necrosis and promoting regeneration[195].

Another fetal associated tissue that has demonstrated to be a potent source of MSC is umbilical cord. Shi et al [196], utilized umbilical cord-derived MSC (UC-MSC) administration to treat acute on chronic liver failure (ACLF) patients that had HBV infection. Twenty four patients were treated with UC-MSCs, and 19 patients were treated with saline as controls. The UC-MSC transfusions significantly increased the survival rates in the patients; diminished MELD score; increased serum albumin, cholinesterase, prothrombin activity; and increased platelet counts. Serum total bilirubin and ALT levels were significantly decreased after the UC-MSC at 48 and 72 weeks.

Fetal liver cells have been utilized clinically for hematopoietic stem cell transplantation with positive safety data. Given the potent proliferative and regenerative activities of these cells in the hepatic setting, a case report was described of a patient who received intrahepatic administration of these cells. Subsequent to administration decrease in MELD score, AST and ALT were reported. At 18 month follow-up MELD score reduced from 18 to 10 [197].

Methods of deriving cord tissue mesenchymal stem cells from human umbilical tissue are provided. The cells are capable of self-renewal and expansion in culture, and have the potential to differentiate into cells of other phenotypes. The method comprises (a) obtaining human umbilical tissue; (b) removing substantially all of blood to yield a substantially blood-free umbilical tissue, (c) dissociating the tissue by mechanical or enzymatic treatment, or both, (d) resuspending the tissue in a culture medium, and (e) providing growth conditions which allow for the growth of a human umbilicus-derived cell capable of self-renewal and expansion in culture and having the potential to differentiate into cells of other phenotypes. Tissue can be obtained from any completed pregnancy, term or less than term, whether delivered vaginally, or through other routes, for example surgical Cesarean section. Obtaining tissue from tissue banks is also considered within the scope of the present invention.

The tissue is rendered substantially free of blood by any means known in the art. For example, the blood can be physically removed by washing, rinsing, and diluting and the like, before or after bulk blood removal for example by suctioning or draining. Other means of obtaining a tissue substantially free of blood cells might include enzymatic or chemical treatment.

Dissociation of the umbilical tissues can be accomplished by any of the various techniques known in the art, including by mechanical disruption, for example, tissue can be aseptically cut with scissors, or a scalpel, or such tissue can be otherwise minced, blended, ground, or homogenized in any manner that is compatible with recovering intact or viable cells from human tissue.

In a presently preferred embodiment, the isolation procedure also utilizes an enzymatic digestion process. Many enzymes are known in the art to be useful for the isolation of individual cells from complex tissue matrices to facilitate growth in culture. As discussed above, a broad range of digestive enzymes for use in cell isolation from tissue is available to the skilled artisan. Ranging from weakly digestive (e.g. deoxyribonucleases and the neutral protease, dispase) to strongly digestive (e.g. papain and trypsin), such enzymes are available commercially. A nonexhaustive list of enzymes compatable herewith includes mucolytic enzyme activities, metalloproteases, neutral proteases, serine proteases (such as trypsin, chymotrypsin, or elastase), and deoxyribonucleases. Presently preferred are enzyme activities selected from metalloproteases, neutral proteases and mucolytic activities. For example, collagenases are known to be useful for isolating various cells from tissues. Deoxyribonucleases can digest single-stranded DNA and can minimize cell-clumping during isolation. Enzymes can be used alone or in combination. Serine protease are preferably used in a sequence following the use of other enzymes as they may degrade the other enzymes being used. The temperature and time of contact with serine proteases must be monitored. Serine proteases may be inhibited with alpha 2 microglobulin in serum and therefore the medium used for digestion is preferably serum-free. EDTA and DNase are commonly used and may improve yields or efficiencies. Preferred methods involve enzymatic treatment with for example collagenase and dispase, or collagenase, dispase, and hyaluronidase, and such methods are provided wherein in certain preferred embodiments, a mixture of collagenase and the neutral protease dispase are used in the dissociating step. More preferred are those methods which employ digestion in the presence of at least one collagenase from Clostridium histolyticum, and either of the protease activities, dispase and thermolysin. Still more preferred are methods employing digestion with both collagenase and dispase enzyme activities. Also preferred are methods which include digestion with a hyaluronidase activity in addition to collagenase and dispase activities. The skilled artisan will appreciate that many such enzyme treatments are known in the art for isolating cells from various tissue sources. For example, the LIBERASE BLENDZYME (Roche) series of enzyme combinations of collagenase and neutral protease are very useful and may be used in the instant methods. Other sources of enzymes are known, and the skilled artisan may also obtain such enzymes directly from their natural sources. The skilled artisan is also well-equipped to assess new, or additional enzymes or enzyme combinations for their utility in isolating the cells of the invention. Preferred enzyme treatments are 0.5, 1, 1.5, or 2 hours long or longer. In other preferred embodiments, the tissue is incubated at 37.degree. C. during the enzyme treatment of the dissociation step. Diluting the digest may also improve yields of cells as cells may be trapped within a viscous digest.

While the use of enzyme activites is presently preferred, it is not required for isolation methods as provided herein. Methods based on mechanical separation alone may be successful in isolating the instant cells from the umbilicus as discussed above.

The cells can be resuspended after the tissue is dissociated into any culture medium as discussed herein above. Cells may be resuspended following a centrifugation step to separate out the cells from tissue or other debris. Resuspension may involve mechanical methods of resuspending, or simply the addition of culture medium to the cells.

Providing the growth conditions allows for a wide range of options as to culture medium, supplements, atmospheric conditions, and relative humidity for the cells. A preferred temperature is 37.degree. C., however the temperature may range from about 35.degree. C. to 39.degree. C. depending on the other culture conditions and desired use of the cells or culture.

Presently preferred are methods which provide cells which require no exogenous growth factors, except as are available in the supplemental serum provided with the Growth Medium. Also provided herein are methods of deriving umbilical cells capable of expansion in the absence of particular growth factors. The methods are similar to the method above, however they require that the particular growth factors (for which the cells have no requirement) be absent in the culture medium in which the cells are ultimately resuspended and grown in. In this sense, the method is selective for those cells capable of division in the absence of the particular growth factors. Preferred cells in some embodiments are capable of growth and expansion in chemically-defined growth media with no serum added. In such cases, the cells may require certain growth factors, which can be added to the medium to support and sustain the cells. Presently preferred factors to be added for growth on serum-free media include one or more of FGF, EGF, IGF, and PDGF. In more preferred embodiments, two, three or all four of the factors are add to serum free or chemically defined media. In other embodiments, LIF is added to serum-free medium to support or improve growth of the cells.

Also provided are methods wherein the cells can expand in the presence of from about 5% to about 20% oxygen in their atmosphere. Methods to obtain cells that require L-valine require that cells be cultured in the presence of L-valine. After a cell is obtained, its need for L-valine can be tested and confirmed by growing on D-valine containing medium that lacks the L-isomer.

Methods are provided wherein the cells can undergo at least 25, 30, 35, or 40 doublings prior to reaching a senescent state. Methods for deriving cells capable of doubling to reach 10.sup.14 cells or more are provided. Preferred are those methods which derive cells that can double sufficiently to produce at least about 10.sup.14, 10.sup.15, 10.sup.16, or 10.sup.17 or more cells when seeded at from about 10.sup.3 to about 10.sup.6 cells/cm.sup.2 in culture. Preferably these cell numbers are produced within 80, 70, or 60 days or less. In one embodiment, cord tissue mesenchymal stem cells are isolated and expanded, and possess one or more markers selected from a group comprising of CD10, CD13, CD44, CD73, CD90, CD141, PDGFr-alpha, or HLA-A,B,C. In addition, the cells do not produce one or more of CD31, CD34, CD45, CD117, CD141, or HLA-DR,DP, DQ.

In one embodiment, bone marrow MSC lots are generated, means of generating BM MSC are known in the literature and examples are incorporated by reference.

In one embodiment BM-MSC are generated as follows

1. 500 mL Isolation Buffer is prepared (PBS+2% FBS+2 mM EDTA) using sterile components or filtering Isolation Buffer through a 0.2 micron filter. Once made, the Isolation Buffer was stored at 2-8.degree. C.
2. The total number of nucleated cells in the BM sample is counted by taking 10.mu.L BM and diluting it 1/50-1/100 with 3% Acetic Acid with Methylene Blue (STEMCELL Catalog #07060). Cells are counted using a hemacytometer.
3. 50 mL Isolation Buffer is warmed to room temperature for 20 minutes prior to use and bone marrow was diluted 5/14 final dilution with room temperature Isolation Buffer (e.g. 25 mL BM was diluted with 45 mL Isolation Buffer for a total volume of 70 mL).
4. In three 50 mL conical tubes (BD Catalog #352070), 17 mL Ficoll-Paque™ PLUS (Catalog #07907/07957) is pipetted into each tube. About 23 mL of the diluted BM from step 3 was carefully layered on top of the Ficoll-Paque™ PLUS in each tube.
5. The tubes are centrifuged at room temperature (15-25.degree. C.) for 30 minutes at 300.times.g in a bench top centrifuge with the brake off.
6. The upper plasma layer is removed and discarded without disturbing the plasma:Ficoll-Paque™ PLUS interface. The mononuclear cells located at the interface layer are carefully removed and placed in a new 50 mL conical tube. Mononuclear cells are resuspended with 40 mL cold (2-8.degree. C.) Isolation Buffer and mixed gently by pipetting.
7. Cells were centrifuged at 300.times.g for 10 minutes at room temperature in a bench top centrifuge with the brake on. The supernatant is removed and the cell pellet resuspended in 1-2 mL cold Isolation Buffer.
8. Cells were diluted 1/50 in 3% Acetic Acid with Methylene Blue and the total number of nucleated cells counted using a hemacytometer.
9. Cells are diluted in Complete Human MesenCult®-Proliferation medium (STEMCELL catalog #05411) at a final concentration of 1.times.10.sup.6 cells/mL.
10. BM-derived cells were ready for expansion and CFU-F assays in the presence of GW2580, which can then be used for specific applications.

In one embodiment, MSC are generated according to protocols previously utilized for treatment of patients utilizing bone marrow derived MSC. Specifically, bone marrow is aspirated (10-30 ml) under local anesthesia (with or without sedation) from the posterior iliac crest, collected into sodium heparin containing tubes and transferred to a Good Manufacturing Practices (GMP) clean room. Bone marrow cells are washed with a washing solution such as Dulbecco's phosphate-buffered saline (DPBS), RPMI, or PBS supplemented with autologous patient plasma and layered on to 25 ml of Percoll (1.073 g/ml) at a concentration of approximately 1-2′107 cells/ml. Subsequently the cells are centrifuged at 900 g for approximately 30 min or a time period sufficient to achieve separation of mononuclear cells from debris and erythrocytes. Said cells are then washed with PBS and plated at a density of approximately 1′106 cells per ml in 175 cm2 tissue culture flasks in DMEM with 10% FCS with flasks subsequently being loaded with a minimum of 30 million bone marrow mononuclear cells. The MSCs are allowed to adhere for 72 h followed by media changes every 3-4 days. Adherent cells are removed with 0.05% trypsin-EDTA and replated at a density of 1′106 per 175 cm2. Said bone marrow MSC may be administered intravenously, or in a preferred embodiment, intrathecally in a patient suffering radiation associated neurodegenerative manifestations. Although doses may be determined by one of skill in the art, and are dependent on various patient characteristics, intravenous administration may be performed at concentrations ranging from 1-10 million MSC per kilogram, with a preferred dose of approximately 2-5 million cells per kilogram.

In order to determine the quality of MSC cultures, flow cytometry is performed on all cultures for surface expression of SH-2, SH-3, SH-4 MSC markers and lack of contaminating CD14- and CD-45 positive cells. Cells were detached with 0.05% trypsin-EDTA, washed with DPBS+2% bovine albumin, fixed in 1% paraformaldehyde, blocked in 10% serum, incubated separately with primary SH-2, SH-3 and SH-4 antibodies followed by PE-conjugated anti-mouse IgG(H+L) antibody. Confluent MSC in 175 cm2 flasks are washed with Tyrode's salt solution, incubated with medium 199 (M199) for 60 min, and detached with 0.05% trypsin-EDTA (Gibco). Cells from 10 flasks were detached at a time and MSCs were resuspended in 40 ml of M199+1% human serum albumin (HSA; American Red Cross, Washington D.C., USA). MSCs harvested from each 10-flask set were stored for up to 4 h at 4° C. and combined at the end of the harvest. A total of 2-10′106 MSC/kg were resuspended in M199+1% HSA and centrifuged at 460 g for 10 min at 20° C. Cell pellets were resuspended in fresh M199+1% HSA media and centrifuged at 460 g for 10 min at 20° C. for three additional times. Total harvest time was 2-4 h based on MSC yield per flask and the target dose. Harvested MSC were cryopreserved in Cryocyte (Baxter, Deerfield, Ill., USA) freezing bags using a rate controlled freezer at a final concentration of 10% DMSO (Research Industries, Salt Lake City, Utah, USA) and 5% HSA. On the day of infusion cryopreserved units were thawed at the bedside in a 37° C. water bath and transferred into 60 ml syringes within 5 min and infused intravenously into patients over 10-15 min. Patients are premedicated with 325-650 mg acetaminophen and 12.5-25 mg of diphenhydramine orally. Blood pressure, pulse, respiratory rate, temperature and oxygen saturation are monitored at the time of infusion and every 15 min thereafter for 3 h followed by every 2 h for 6 h.

Within the context of the invention, exosomes and microparticles may be used interchangeably. Exosomes from MSC may be generated from a mesenchymal stem cell conditioned medium (MSC-CM). Said exosomes are used in the context of the invention to reprogram immunocytes ex vivo or in vivo. Said particle may be isolated for example by being separated from non-associated components based on any property of the particle. For example, the particle may be isolated based on molecular weight, size, shape, composition or biological activity. The conditioned medium may be filtered or concentrated or both during, prior to or subsequent to separation. For example, it may be filtered through a membrane, for example one with a size or molecular weight cut-off. It may be subject to tangential force filtration or ultrafiltration. Filtration of conditioned media is described in the following and incorporated by reference [198]. For example, filtration with a membrane of a suitable molecular weight or size cutoff. The conditioned medium, optionally filtered or concentrated or both, may be subject to further separation means, such as column chromatography. For example, high performance liquid chromatography (HPLC) with various columns may be used. The columns may be size exclusion columns or binding columns. One or more properties or biological activities of the particle may be used to track its activity during fractionation of the mesenchymal stem cell conditioned medium (MSC-CM). As an example, light scattering, refractive index, dynamic light scattering or UV-visible detectors may be used to follow the particles. For example, a therapeutic activity such as antirheumatic activity may be used to track the activity during fractionation. In one embodiment antirheumatic activity is assessed by ability to inhibit TNF-alpha production from stimulated monocytes or monocytic lineage cell such as macrophages or dendritic cells.

In one aspect of the invention MSC are cultured for about 1, 2, 3, 4, 5, 6, 7, 8, 9, 10 days or more, for example 3 days. The conditioned medium may be obtained by separating the cells from the medium. The conditioned medium may be centrifuged, for example at 500 g. it may be concentrated by filtration through a membrane. The membrane may comprise a >1000 kDa membrame. The conditioned medium may be concentrated about 50 times or more. The conditioned medium may be subject to liquid chromatography such as HPLC. The conditioned medium may be separated by size exclusion. Any size exclusion matrix such as Sepharose may be used. As an example, a TSK Guard column SWXL, 6.times.40 mm or a TSK gel G4000 SWXL, 7.8.times.300 mm may be employed. The eluent buffer may comprise any physiological medium such as saline. It may comprise 20 mM phosphate buffer with 150 mM of NaCl at pH 7.2. The chromatography system may be equilibrated at a flow rate of 0.5 ml/min. The elution mode may be isocratic. UV absorbance at 220 nm may be used to track the progress of elution. Fractions may be examined for dynamic light scattering (DLS) using a quasi-elastic light scattering (QELS) detector. Fractions which are found to exhibit dynamic light scattering may be retained. For example, a fraction which is produced by the general method as described above, and which elutes with a retention time of 11-13 minutes, such as 12 minutes, is found to exhibit dynamic light scattering. The r.sub.h of particles in this peak is about 45-55 nm. Such fractions comprise mesenchymal stem cell particles such as exosomes.

MSC can be prepared from a variety of tissues, such as bone marrow cells [199-205], umbilical cord tissue [206-208], peripheral blood [209-211], amniotic membrane [212], amniotic fluid, mobilized peripheral blood [213], adipose tissue [214, 215], endometrium and other tissues. When tissue sources of MSC are used said tissue isolates from which the Reprogrammed immune cells are isolated comprise a mixed populations of cells. Reprogrammed immune cells constitute a very small percentage in these initial populations. They must be purified away from the other cells before they can be expanded in culture sufficiently to obtain enough cells for therapeutic applications.

The choice of formulation for administering reprogrammed immune cells for a given application will depend on a variety of factors. Prominent among these will be the species of subject, the nature of the disorder, dysfunction, or disease being treated and its state and distribution in the subject, the nature of other therapies and agents that are being administered, the optimum route for administration of the reprogrammed immune cells, survivability of reprogrammed immune cells via the route, the dosing regimen, and other factors that will be apparent to those skilled in the art. In particular, for instance, the choice of suitable carriers and other additives will depend on the exact route of administration and the nature of the particular dosage form, for example, liquid dosage form (e.g., whether the composition is to be formulated into a solution, a suspension, gel or another liquid form, such as a time release form or liquid-filled form).

For example, cell survival can be an important determinant of the efficacy of cell-based therapies. This is true for both primary and adjunctive therapies. Another concern arises when target sites are inhospitable to cell seeding and cell growth. This may impede access to the site and/or engraftment there of therapeutic Reprogrammed immune cells. Various embodiments of the invention comprise measures to increase cell survival and/or to overcome problems posed by barriers to seeding and/or growth.

Examples of compositions comprising reprogrammed immune cells include liquid preparations, including suspensions and preparations for intramuscular or intravenous administration (e.g., injectable administration), such as sterile suspensions or emulsions. Such compositions may comprise an admixture of Reprogrammed immune cells with a suitable carrier, diluent, or excipient such as sterile water, physiological saline, glucose, dextrose, or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE,” 17th edition, 1985, incorporated herein by reference, may be consulted to prepare suitable preparations, without undue experimentation.

Compositions of the invention often are conveniently provided as liquid preparations, e.g., isotonic aqueous solutions, suspensions, emulsions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues.

Various additives often will be included to enhance the stability, sterility, and isotonicity of the compositions, such as antimicrobial preservatives, antioxidants, chelating agents, and buffers, among others. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents that delay absorption, for example, aluminum monostearate, and gelatin. According to the present invention, however, any vehicle, diluent, or additive used would have to be compatible with the cells.

Reprogrammed immune cells solutions, suspensions, and gels normally contain a major amount of water (preferably purified, sterilized water) in addition to the cells. Minor amounts of other ingredients such as pH adjusters (e.g., a base such as NaOH), emulsifiers or dispersing agents, buffering agents, preservatives, wetting agents and jelling agents (e.g., methylcellulose) may also be present.

In some embodiments of the invention, treatment of viral associated liver damage is disclosed using immune cells that have been reprogrammed with regenerative cells. Some patients hospitalized for COVID-19 have had increased levels of liver enzymes like alanine aminotransferase (ALT) and aspartate aminotransferase (AST) that indicate their livers are at least temporarily damaged. Also, liver damage is more common in patients who have severe COVID-19 disease. [00384] ypically, the compositions will be isotonic, i.e., they will have the same osmotic pressure as blood and lacrimal fluid when properly prepared for administration.

The desired isotonicity of the compositions of this invention may be accomplished using sodium chloride, or other pharmaceutically acceptable agents such as dextrose, boric acid, sodium tartrate, propylene glycol, or other inorganic or organic solutes. Sodium chloride is preferred particularly for buffers containing sodium ions.

Viscosity of the compositions, if desired, can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount, which will achieve the selected viscosity. Viscous compositions are normally prepared from solutions by the addition of such thickening agents.

A pharmaceutically acceptable preservative or cell stabilizer can be employed to increase the life of reprogrammed immune cells compositions. If such preservatives are included, it is well within the purview of the skilled artisan to select compositions that will not affect the viability or efficacy of the reprogrammed immune cells.

Those skilled in the art will recognize that the components of the compositions should be chemically inert. This will present no problem to those skilled in chemical and pharmaceutical principles. Problems can be readily avoided by reference to standard texts or by simple experiments (not involving undue experimentation) using information provided by the disclosure, the documents cited herein, and generally available in the art.

Sterile injectable solutions can be prepared by incorporating the cells utilized in practicing the present invention in the required amount of the appropriate solvent with various amounts of the other ingredients, as desired.

In some embodiments, reprogrammed immune cells are formulated in a unit dosage injectable form, such as a solution, suspension, or emulsion. Pharmaceutical formulations suitable for injection of Reprogrammed immune cells typically are sterile aqueous solutions and dispersions. Carriers for injectable formulations can be a solvent or dispersing medium containing, for example, water, saline, phosphate buffered saline, polyol (for example, glycerol, propylene glycol, liquid polyethylene glycol, and the like), and suitable mixtures thereof.

The skilled artisan can readily determine the amount of cells and optional additives, vehicles, and/or carrier in compositions to be administered in methods of the invention. Typically, any additives (in addition to the cells) are present in an amount of 0.001 to 50 wt % in solution, such as in phosphate buffered saline. The active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %.

For any composition to be administered to an animal or human, and for any particular method of administration, it is preferred to determine therefore: toxicity, such as by determining the lethal dose (LD) and LD50 in a suitable animal model, e.g., rodent such as mouse or rat; and, the dosage of the composition(s), concentration of components therein, and timing of administering the composition(s), which elicit a suitable response. Such determinations do not require undue experimentation from the knowledge of the skilled artisan, this disclosure, and the documents cited herein. And, the time for sequential administrations can be ascertained without undue experimentation.

In some embodiments Reprogrammed immune cells are encapsulated for administration, particularly where encapsulation enhances the effectiveness of the therapy, or provides advantages in handling and/or shelf life. Encapsulation in some embodiments where it increases the efficacy of REPROGRAMMED IMMUNE CELLS mediated immunosuppression may, as a result, also reduce the need for immunosuppressive drug therapy.

Also, encapsulation in some embodiments provides a barrier to a subject's immune system that may further reduce a subject's immune response to the Reprogrammed immune cells (which generally are not immunogenic or are only weakly immunogenic in allogeneic transplants), thereby reducing any graft rejection or inflammation that might occur upon administration of the cells.

In a variety of embodiments where reprogrammed immune cells are administered in admixture with cells of another type, which are more typically immunogenic in an allogeneic or xenogeneic setting, encapsulation may reduce or eliminate adverse host immune responses to the non-reprogrammed immune cells cells and/or GVHD that might occur in an immunocompromised host if the admixed cells are immunocompetent and recognize the host as non-self.

Reprogrammed immune cells may be encapsulated by membranes, as well as capsules, prior to implantation. It is contemplated that any of the many methods of cell encapsulation available may be employed. In some embodiments, cells are individually encapsulated. In some embodiments, many cells are encapsulated within the same membrane. In embodiments in which the cells are to be removed following implantation, a relatively large size structure encapsulating many cells, such as within a single membrane, may provide a convenient means for retrieval.

A wide variety of materials may be used in various embodiments for microencapsulation of Reprogrammed immune cells. Such materials include, for example, polymer capsules, alginate-poly-L-lysine-alginate microcapsules, barium poly-L-lysine alginate capsules, barium alginate capsules, polyacrylonitrile/polyvinylchloride (PAN/PVC) hollow fibers, and polyethersulfone (PES) hollow fibers.

Techniques for microencapsulation of cells that may be used for administration of Reprogrammed immune cells are known to those of skill in the art and are described, for example, in Chang, P., et al., 1999; Matthew, H. W., et al., 1991; Yanagi, K., et al., 1989; Cai Z. H., et al., 1988; Chang, T. M., 1992 and in U.S. Pat. No. 5,639,275 (which, for example, describes a biocompatible capsule for long-term maintenance of cells that stably express biologically active molecules. Additional methods of encapsulation are in European Patent Publication No. 301,777 and U.S. Pat. Nos. 4,353,888; 4,744,933; 4,749,620; 4,814,274; 5,084,350; 5,089,272; 5,578,442; 5,639,275; and 5,676,943. All of the foregoing are incorporated herein by reference in parts pertinent to encapsulation of Reprogrammed immune cells.

Certain embodiments incorporate Reprogrammed immune cells into a polymer, such as a biopolymer or synthetic polymer. Examples of biopolymers include, but are not limited to, fibronectin, fibin, fibrinogen, thrombin, collagen, and proteoglycans. Other factors, such as the cytokines discussed above, can also be incorporated into the polymer. In other embodiments of the invention, Reprogrammed immune cells may be incorporated in the interstices of a three-dimensional gel. A large polymer or gel, typically, will be surgically implanted. A polymer or gel that can be formulated in small enough particles or fibers can be administered by other common, more convenient, non-surgical routes.

Pharmaceutical compositions of the invention may be prepared in many forms that include tablets, hard or soft gelatin capsules, aqueous solutions, suspensions, and liposomes and other slow-release formulations, such as shaped polymeric gels. Oral liquid pharmaceutical compositions may be in the form of, for example, aqueous or oily suspensions, solutions, emulsions, syrups, or elixirs, or may be presented as a dry product for constitution with water or other suitable vehicle before use. Such liquid pharmaceutical compositions may contain conventional additives such as suspending agents, emulsifying agents, non-aqueous vehicles (which may include edible oils), or preservatives. An oral dosage form may be formulated such that cells are released into the intestine after passing through the stomach. Such formulations are described in U.S. Pat. No. 6,306,434 and in the references contained therein.

Pharmaceutical compositions suitable for rectal administration can be prepared as unit dose suppositories. Suitable carriers include saline solution and other materials commonly used in the art.

For administration by inhalation, cells can be conveniently delivered from an insufflator, nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide, or other suitable gas. In the case of a pressurized aerosol, the dosage unit may be determined by providing a valve to deliver a metered amount.

Alternatively, for administration by inhalation or insufflation, a means may take the form of a dry powder composition, for example, a powder mix of a modulator and a suitable powder base such as lactose or starch. The powder composition may be presented in unit dosage form in, for example, capsules or cartridges or, e.g., gelatin or blister packs from which the powder may be administered with the aid of an inhalator or insufflator. For intra-nasal administration, cells may be administered via a liquid spray, such as via a plastic bottle atomizer.

Reprogrammed immune cells may be administered with other pharmaceutically active agents. In some embodiments one or more of such agents are formulated together with Reprogrammed immune cells for administration. In some embodiments the Reprogrammed immune cells and the one or more agents are in separate formulations. In some embodiments the compositions comprising the Reprogrammed immune cells and/or the one or more agents are formulated with regard to adjunctive use with one another.

Reprogrammed immune cells may be administered in a formulation comprising a immunosuppressive agents, such as any combination of any number of a corticosteroid, cyclosporin A, a cyclosporin-like immunosuppressive agent, cyclophosphamide, antithymocyte globulin, azathioprine, rapamycin, FK-506, and a macrolide-like immunosuppressive agent other than FK-506 and rapamycin. In certain embodiments, such agents include a corticosteroid, cyclosporin A, azathioprine, cyclophosphamide, rapamycin, and/or FK-506. Immunosuppressive agents in accordance with the foregoing may be the only such additional agents or may be combined with other agents, such as other agents noted herein. Other immunosuppressive agents include Tacrolimus, Mycophenolate mofetil, and Sirolimus.

Such agents also include antibiotic agents, antifungal agents, and antiviral agents, to name just a few other pharmacologically active substances and compositions that may be used in accordance with embodiments of the invention.

Typical antibiotics or anti-mycotic compounds include, but are not limited to, penicillin, streptomycin, amphotericin, ampicillin, gentamicin, kanamycin, mycophenolic acid, nalidixic acid, neomycin, nystatin, paromomycin, polymyxin, puromycin, rifampicin, spectinomycin, tetracycline, tylosin, zeocin, and cephalosporins, aminoglycosides, and echinocandins.

Further additives of this type relate to the fact that Reprogrammed immune cells, like other stems cells, following administration to a subject may “home” to an environment favorable to their growth and function. Such “homing” often concentrates the cells at sites where they are needed, such as sites of immune disorder, dysfunction, or disease. A number of substances are known to stimulate homing. They include growth factors and trophic signaling agents, such as cytokines. They may be used to promote homing of Reprogrammed immune cells to therapeutically targeted sites. They may be administered to a subject prior to treatment with Reprogrammed immune cells, together with reprogrammed immune cells, or after reprogrammed immune cells are administered.

Certain cytokines, for instance, alter or affect the migration of reprogrammed immune cells or their differentiated counterparts to sites in need of therapy, such as immunocompromised sites. Cytokines that may be used in this regard include, but are not limited to, stromal cell derived factor-1 (SDF-1), stem cell factor (SCF), angiopoietin-1, placenta-derived growth factor (PIGF), granulocyte-colony stimulating factor (G-CSF), cytokines that stimulate expression of endothelial adhesion molecules such as ICAMs and VCAMs, and cytokines that engender or facilitate homing.

They may be administered to a subject as a pre-treatment, along with Reprogrammed immune cells, or after reprogrammed immune cells have been administered, to promote homing to desired sites and to achieve improved therapeutic effect, either by improved homing or by other mechanisms. Such factors may be combined with Reprogrammed immune cells in a formulation suitable for them to be administered together. Alternatively, such factors may be formulated and administered separately.

Order of administration, formulations, doses, frequency of dosing, and routes of administration of factors (such as the cytokines discussed above) and Reprogrammed immune cells generally will vary with the disorder or disease being treated, its severity, the subject, other therapies that are being administered, the stage of the disorder or disease, and prognostic factors, among others. General regimens that have been established for other treatments provide a framework for determining appropriate dosing in reprogrammed immune cells-mediated direct or adjunctive therapy. These, together with the additional information provided herein, will enable the skilled artisan to determine appropriate administration procedures in accordance with embodiments of the invention, without undue experimentation.

Reprogrammed immune cells can be administered to a subject by any of a variety of routes known to those skilled in the art that may be used to administer cells to a subject.

Among methods that may be used in this regard in embodiments of the invention are methods for administering reprogrammed immune cells by a parenteral route. Parenteral routes of administration useful in various embodiments of the invention include, among others, administration by intravenous, intraarterial, intracardiac, intraspinal, intrathecal, intraosseous, intraarticular, intrasynovial, intracutaneous, intradermal, subcutaneous, and/or intramuscular injection. In some embodiments intravenous, intraarterial, intracutaneous, intradermal, subcutaneous and/or intramuscular injection are used. In some embodiments intravenous, intraarterial, intracutaneous, subcutaneous, and/or intramuscular injection are used.

In various embodiments of the invention reprogrammed immune cells are administered by systemic injection. Systemic injection, such as intravenous injection, offers one of the simplest and least invasive routes for administering reprogrammed immune cells. In some cases, these routes may require high reprogrammed immune cells doses for optimal effectiveness and/or homing by the reprogrammed immune cells to the target sites. In a variety of embodiments reprogrammed immune cells may be administered by targeted and/or localized injections to ensure optimum effect at the target sites.

Reprogrammed immune cells may be administered to the subject through a hypodermic needle by a syringe in some embodiments of the invention. In various embodiments, reprogrammed immune cells are administered to the subject through a catheter. In a variety of embodiments, reprogrammed immune cells are administered by surgical implantation. Further in this regard, in various embodiments of the invention, Reprogrammed immune cells are administered to the subject by implantation using an arthroscopic procedure. In some embodiments Reprogrammed immune cells are administered to the subject in or on a solid support, such as a polymer or gel. In various embodiments, Reprogrammed immune cells are administered to the subject in an encapsulated form.

In additional embodiments of the invention, Reprogrammed immune cells are suitably formulated for oral, rectal, epicutaneous, ocular, nasal, and/or pulmonary delivery and are administered accordingly.

Compositions can be administered in dosages and by techniques well known to those skilled in the medical and veterinary arts taking into consideration such factors as the age, sex, weight, and condition of the particular patient, and the formulation that will be administered (e.g., solid vs. liquid). Doses for humans or other mammals can be determined without undue experimentation by the skilled artisan, from this disclosure, the documents cited herein, and the knowledge in the art.

The dose of reprogrammed immune cells appropriate to be used in accordance with various embodiments of the invention will depend on numerous factors. It may vary considerably for different circumstances. The parameters that will determine optimal doses of reprogrammed immune cells to be administered for primary and adjunctive therapy generally will include some or all of the following: the disease being treated and its stage; the species of the subject, their health, gender, age, weight, and metabolic rate; the subject's immunocompetence; other therapies being administered; and expected potential complications from the subject's history or genotype. The parameters may also include: whether the Reprogrammed immune cells are syngeneic, autologous, allogeneic, or xenogeneic; their potency (specific activity); the site and/or distribution that must be targeted for the Reprogrammed immune cells to be effective; and such characteristics of the site such as accessibility to Reprogrammed immune cells and/or engraftment of Reprogrammed immune cells. Additional parameters include co-administration with Reprogrammed immune cells of other factors (such as growth factors and cytokines). The optimal dose in a given situation also will take into consideration the way in which the cells are formulated, the way they are administered, and the degree to which the cells will be localized at the target sites following administration. Finally, the determination of optimal dosing necessarily will provide an effective dose that is neither below the threshold of maximal beneficial effect nor above the threshold where the deleterious effects associated with the dose of Reprogrammed immune cells outweighs the advantages of the increased dose.

The optimal dose of reprogrammed immune cells for some embodiments will be in the range of doses used for autologous, mononuclear bone marrow transplantation. For fairly pure preparations of reprogrammed immune cells, optimal doses in various embodiments will range from 10.sup.4 to 10.sup.8 reprogrammed immune cells cells/kg of recipient mass per administration. In some embodiments the optimal dose per administration will be between 10.sup.5 to 10.sup.7 reprogrammed immune cells cells/kg. In many embodiments the optimal dose per administration will be 5.times.10.sup.5 to 5.times.10.sup.6 reprogrammed immune cells cells/kg. By way of reference, higher doses in the foregoing are analogous to the doses of nucleated cells used in autologous mononuclear bone marrow transplantation. Some of the lower doses are analogous to the number of CD34.sup.+ cells/kg used in autologous mononuclear bone marrow transplantation.

It is to be appreciated that a single dose may be delivered all at once, fractionally, or continuously over a period of time. The entire dose also may be delivered to a single location or spread fractionally over several locations.

In various embodiments, Reprogrammed immune cells may be administered in an initial dose, and thereafter maintained by further administration of Reprogrammed immune cells. Reprogrammed immune cells may be administered by one method initially, and thereafter administered by the same method or one or more different methods. The subject's MSC levels can be maintained by the ongoing administration of the cells. Various embodiments administer the Reprogrammed immune cells either initially or to maintain their level in the subject or both by intravenous injection. In a variety of embodiments, other forms of administration, are used, dependent upon the patient's condition and other factors, discussed elsewhere herein.

It is noted that human subjects are treated generally longer than experimental animals; but, treatment generally has a length proportional to the length of the disease process and the effectiveness of the treatment. Those skilled in the art will take this into account in using the results of other procedures carried out in humans and/or in animals, such as rats, mice, non-human primates, and the like, to determine appropriate doses for humans. Such determinations, based on these considerations and taking into account guidance provided by the present disclosure and the prior art will enable the skilled artisan to do so without undue experimentation.

Suitable regimens for initial administration and further doses or for sequential administrations may all be the same or may be variable. Appropriate regiments can be ascertained by the skilled artisan, from this disclosure, the documents cited herein, and the knowledge in the art.

The dose, frequency, and duration of treatment will depend on many factors, including the nature of the disease, the subject, and other therapies that may be administered. Accordingly, a wide variety of regimens may be used to administer Reprogrammed immune cells.

In some embodiments Reprogrammed immune cells are administered to a subject in one dose. In others Reprogrammed immune cells are administered to a subject in a series of two or more doses in succession. In some other embodiments wherein Reprogrammed immune cells are administered in a single dose, in two doses, and/or more than two doses, the doses may be the same or different, and they are administered with equal or with unequal intervals between them.

Reprogrammed immune cells may be administered in many frequencies over a wide range of times. In some embodiments, reprogrammed immune cells are administered over a period of less than one day. In other embodiment they are administered over two, three, four, five, or six days. In some embodiments Reprogrammed immune cells are administered one or more times per week, over a period of weeks. In other embodiments they are administered over a period of weeks for one to several months. In various embodiments they may be administered over a period of months. In others they may be administered over a period of one or more years. Generally lengths of treatment will be proportional to the length of the disease process, the effectiveness of the therapies being applied, and the condition and response of the subject being treated.

The immunomodulatory properties of reprogrammed immune cells may be used in treating a wide variety of disorders, dysfunctions and diseases, such as those that, intrinsically, as a secondary effect or as a side effect of treatment, present with deleterious immune system processes and effects. Several illustrations are discussed below.

In a variety of embodiments involving transplant therapies, reprogrammed immune cells can be used alone for an immunosuppressive purpose, or together with other agents. Reprogrammed immune cells can be administered before, during, or after one or more transplants. If administered during transplant, reprogrammed immune cells can be administered separately or together with transplant material. If separately administered, the Reprogrammed immune cells can be administered sequentially or simultaneously with the other transplant materials. Furthermore, Reprogrammed immune cells may be administered well in advance of the transplant and/or well after, alternatively to or in addition to administration at or about the same time as administration of the transplant.

Other agents that can be used in conjunction with reprogrammed immune cells, in transplantation therapies in particular, include immunomodulatory agents, such as those described elsewhere herein, particularly immunosuppressive agents, more particularly those described elsewhere herein, especially in this regard, one or more of a corticosteroid, cyclosporin A, a cyclosporin-like immunosuppressive compound, azathioprine, cyclophosphamide, methotrexate, and an immunosuppressive monoclonal antibody agent.

Reprogrammed immune cells can modulate immune responses. In particular in this regard, it has been found that Reprogrammed immune cells can suppress immune responses, including but not limited to immune responses involved in, for example, HVG response and GVHD, to name just two. In an even more detailed particular in this regard, it has been found that Reprogrammed immune cells can suppress proliferation of T-cells, even in the presence of potent T-cell stimulators, such as Concanavalin A and allogeneic or xenogeneic stimulator cells.

Moreover, it has been found that even relatively small amounts of reprogrammed immune cells can suppress these responses. Indeed, only 3% Reprogrammed immune cells in mixed lymphocyte reactions is sufficient to reduce T-cell response by 50% in vitro.

Embodiments of the invention relate to using reprogrammed immune cells immunomodulation to treat an immune dysfunction, disorder, or disease, either solely, or as an adjunctive therapy. Embodiments in this regard relate to congenital immune deficiencies and autoimmune dysfunctions, disorders, and diseases. Various embodiments relate, in this regard, to using Reprogrammed immune cells to treat, solely or adjunctively, Crohn's disease, Guillain-Barre syndrome, lupus erythematosus (also called “SLE” and systemic lupus erythematosus), multiple sclerosis, myasthenia gravis, optic neuritis, psoriasis, rheumatoid arthritis, Graves' disease, Hashimoto's disease, Ord's thyroiditis, diabetes mellitus (type 1), Reiter's syndrome, autoimmune hepatitis, primary biliary cirrhosis, antiphospholipid antibody syndrome (“APS”), opsoclonus-myoclonus syndrome (“OMS”), temporal arteritis, acute disseminated encephalomyelitis (“ADEM” and “ADE”), Goodpasture's, syndrome, Wegener's granulomatosis, celiac disease, pemphigus, polyarthritis, autism, autism spectrum disorder, post traumatic stress disorder, and warm autoimmune hemolytic anemia.

Particular embodiments among these relate to Crohn's disease, lupus erythematosus (also called “SLE” and systemic lupus erythematosus), multiple sclerosis, myasthenia gravis, psoriasis, rheumatoid arthritis, Graves' disease, Hashimoto's disease, diabetes mellitus (type 1), Reiter's syndrome, primary biliary cirrhosis, celiac disease, polyarhritis, and warm autoimmune hemolytic anemia.

In addition, reprogrammed immune cells are used in a variety of embodiments in this regard, solely and, typically, adjunctively, to treat a variety of diseases thought to have an autoimmune component, including but not limited to embodiments that may be used to treat endometriosis, interstitial cystitis, neuromyotonia, scleroderma, progressive systemic scleroderma, vitiligo, vulvodynia, Chagas' disease, sarcoidosis, chronic fatigue syndrome, and dysautonomia.

In one embodiment higher expression of one or more proteins selected from a group comprising of TNFR-1, ENA-78, and IGFBP-3 is utilized to select MSC generated from MSC progenitors and/or pluripotent cells

Inherited immune system disorders include Severe Combined Immunodeficiency (SCID) including but not limited to SCID with Adenosine Deaminase Deficiency (ADA-SCID), SCID which is X-linked, SCID with absence of T & B Cells, SCID with absence of T Cells, Normal B Cells, Omenn Syndrome, Neutropenias including but not limited to Kostmann Syndrome, Myelokathexis; Ataxia-Telangiectasia, Bare Lymphocyte Syndrome, Common Variable Immunodeficiency, DiGeorge Syndrome, Leukocyte Adhesion Deficiency; and phagocyte Disorders (phagocytes are immune system cells that can engulf and kill foreign organisms) including but not limited to Chediak-Higashi Syndrome, Chronic Granulomatous Disease, Neutrophil Actin Deficiency, Reticular Dysgenesis. Reprogrammed immune cells may be administered adjunctively to a treatment for any of the foregoing diseases.

In one embodiment tissue culture supernatant is derived from cultures of reprogrammed immune cells and utilized for therapeutic applications. Use of tissue culture supernatant is described in the following patents and incorporated by reference U.S. Pat. Nos. 8,703,710; 9,192,632; 6,642,048; 7,790,455; 9,192,632; and the following patent applications; 20160022738; 20160000699; 20150024483; 20130251670; 20120294949; 20120276215; 20120195969; 20110293583; 20110171182; 20110129447; 20100159588; 20080241112.

### Example 1: Treatment of Liver Failure with Reprogrammed T Cells

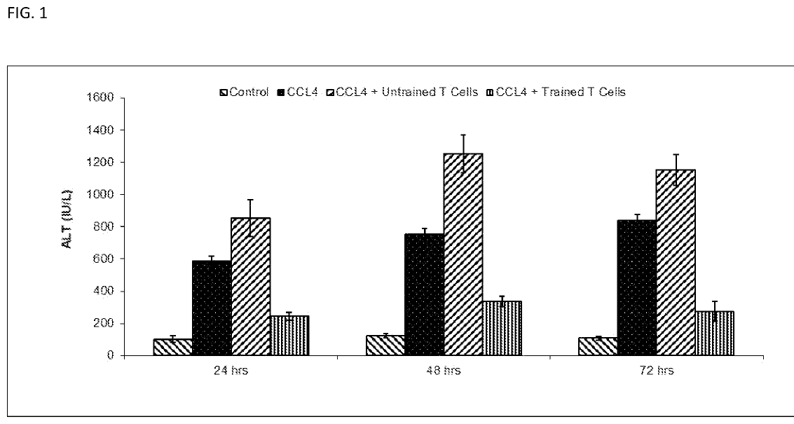

Forty BALB/c mice were randomly assigned to the following 4 groups:
(1) Normal control group: mice first receiving intraperitoneal (i.p.) injection of corn oil were then injected with 200 μl PBS intravenously 30 min later (Control).
(2) Carbon tetrachloride group: mice first receiving i.p. injection of a single dose of CCl4 (Sigma-aldrich, St Louis, United States) for induction of acute liver injury were injected 200 μl PBS intravenously 30 min later. (CCL4)
(3) Unprogrammed T Cells: mice first receiving i.p. injection of CCl4 were injected intravenously with 1×10(6) BALB/C T cells suspended in 200 μl of PBS 30 min later. Mice were sacrificed 24 h after injection of CCl4, and blood was collected.
(4) Programmed T Cells: mice first receiving i.p. injection of CCl4 were injected intravenously with 1×106 BALB/C T cells that were “programmed” by culture with C57/BL6 bone marrow MSC. These cells were admixed at a one to one ratio with BALB/c T cells and cultured at 20 IU/ml interleukin 2. Concentration of T cells and BM-MSC in culture was 500,000 cells per ml. After 48 hours of culture T cells were purified by gently removing the non-adherent fraction. 1 million T cells were suspended in 200 μl of PBS and injected. Mice were evaluated for ALT at 24, 48 and 72 hours. Results are shown in FIG. 1.

### REFERENCES

1. Kelso, L. A., Cirrhosis: caring for patients with end-stage liver failure. Nurse Pract, 2008. 33(7): p. 24-30; quiz 30-1.
2. Bernuau, J., B. Rueff, and J. P. Benhamou, Fulminant and subfulminant liver failure: definitions and causes. Semin Liver Dis, 1986. 6(2): p. 97-106.
3. Farci, P., et al., Hepatitis C virus-associated fulminant hepatic failure. N Engl J Med, 1996. 335(9): p. 631-4.
4. Navarro, V. J. and J. R. Senior, Drug-related hepatotoxicity. N Engl J Med, 2006. 354(7): p. 731-9.
5. Sargent, S., Management of patients with advanced liver cirrhosis. Nurs Stand, 2006. 21(11): p. 48-56; quiz 58.
6. Kisseleva, T., E. Gigante, and D. A. Brenner, Recent advances in liver stem cell therapy. Curr Opin Gastroenterol, 2010. 26(4): p. 395-402.
7. Wu, Y. M., et al., Hepatocyte transplantation and drug-induced perturbations in liver cell compartments. Hepatology, 2008. 47(1): p. 279-87.
8. Fausto, N., J. S. Campbell, and K. J. Riehle, Liver regeneration. Hepatology, 2006. 43(2 Suppl 1): p. S45-53.
9. Michalopoulos, G. K., Liver regeneration: alternative epithelial pathways. Int J Biochem Cell Biol, 2011. 43(2): p. 173-9.
10. Clavien, P. A., et al., Strategies for safer liver surgery and partial liver transplantation. N Engl J Med, 2007. 356(15): p. 1545-59.
11. Adam, R., et al., Two-stage hepatectomy: A planned strategy to treat irresectable liver tumors. Ann Surg, 2000. 232(6): p. 777-85.
12. Brown, R. S., Jr., Live donors in liver transplantation. Gastroenterology, 2008. 134(6): p. 1802-13.
13. Michalopoulos, G. K., Principles of liver regeneration and growth homeostasis. Compr Physiol, 2013. 3(1): p. 485-513.
14. Van Thiel, D. H., et al., Rapid growth of an intact human liver transplanted into a recipient larger than the donor. Gastroenterology, 1987. 93(6): p. 1414-9.
15. Cressman, D. E., et al., Liver failure and defective hepatocyte regeneration in interleukin-6-deficient mice. Science, 1996. 274(5291): p. 1379-83.
16. Malato, Y., et al., Hepatocyte-specific inhibitor-of-kappaB-kinase deletion triggers the innate immune response and promotes earlier cell proliferation during liver regeneration. Hepatology, 2008. 47(6): p. 2036-50.
17. Li, M., et al., Study on the activity of the signaling pathways regulating hepatocytes from G0 phase into G1 phase during rat liver regeneration. Cell Mol Biol Lett, 2014. 19(2): p. 181-200.
18. Fan, G., et al., Modulation of retinoblastoma and retinoblastoma-related proteins in regenerating rat liver and primary hepatocytes. Cell Growth Differ, 1995. 6(11): p. 1463-76.
19. Spiewak Rinaudo, J. A. and S. S. Thorgeirsson, Detection of a tyrosine-phosphorylated form of cyclin A during liver regeneration. Cell Growth Differ, 1997. 8(3): p. 301-9.
20. DeLeve, L. D., Liver sinusoidal endothelial cells and liver regeneration. J Clin Invest, 2013. 123(5): p. 1861-6.
21. Maher, J. J., Cell-specific expression of hepatocyte growth factor in liver. Upregulation in sinusoidal endothelial cells after carbon tetrachloride. J Clin Invest, 1993. 91(5): p. 2244-52.
22. Matsumoto, K., et al., Serial changes of serum growth factor levels and liver regeneration after partial hepatectomy in healthy humans. Int J Mol Sci, 2013. 14(10): p. 20877-89.
23. Nakamura, T., et al., Hepatocyte growth factor twenty years on: Much more than a growth factor. J Gastroenterol Hepatol, 2011. 26 Suppl 1: p. 188-202.
24. Mars, W. M., et al., Immediate early detection of urokinase receptor after partial hepatectomy and its implications for initiation of liver regeneration. Hepatology, 1995. 21(6): p. 1695-701.
25. Shanmukhappa, K., et al., Urokinase-type plasminogen activator supports liver repair independent of its cellular receptor. BMC Gastroenterol, 2006. 6: p. 40.
26. Borowiak, M., et al., Met provides essential signals for liver regeneration. Proc Natl Acad Sci USA, 2004. 101(29): p. 10608-13.
27. Natarajan, A., B. Wagner, and M. Sibilia, The EGF receptor is required for efficient liver regeneration. Proc Natl Acad Sci USA, 2007. 104(43): p. 17081-6.
28. Nygard, I. E., et al., The genetic regulation of the terminating phase of liver regeneration. Comp Hepatol, 2012. 11(1): p. 3.
29. Mosser, D. M. and X. Zhang, Interleukin-10: new perspectives on an old cytokine. Immunol Rev, 2008. 226: p. 205-18.
30. Yin, S., et al., Enhanced liver regeneration in IL-10-deficient mice after partial hepatectomy via stimulating inflammatory response and activating hepatocyte STAT3. Am J Pathol, 2011. 178(4): p. 1614-21.
31. Miyaoka, Y. and A. Miyajima, To divide or not to divide: revisiting liver regeneration. Cell Div, 2013. 8(1): p. 8.
32. Farber, E., Similarities in the sequence of early histological changes induced in the liver of the rat by ethionine, 2-acetylamino-fluorene, and 3′-methyl-4-dimethylaminoazobenzene. Cancer Res, 1956. 16(2): p. 142-8.
33. Evarts, R. P., et al., A precursor-product relationship exists between oval cells and hepatocytes in rat liver. Carcinogenesis, 1987. 8(11): p. 1737-40.
34. Libbrecht, L. and T. Roskams, Hepatic progenitor cells in human liver diseases. Semin Cell Dev Biol, 2002. 13(6): p. 389-96.
35. Banas, A., et al., Adipose tissue-derived mesenchymal stem cells as a source of human hepatocytes. Hepatology, 2007. 46(1): p. 219-28.
36. Petersen, B. E., et al., Bone marrow as a potential source of hepatic oval cells. Science, 1999. 284(5417): p. 1168-70.
37. Margini, C., et al., Bone marrow derived stem cells for the treatment of end-stage liver disease. World J Gastroenterol, 2014. 20(27): p. 9098-9105.
38. Theise, N. D., et al., Liver from bone marrow in humans. Hepatology, 2000. 32(1): p. 11-6.
39. Filippi, C. and A. Dhawan, Current status of human hepatocyte transplantation and its potentialfor Wilson's disease. Ann N Y Acad Sci, 2014. 1315: p. 50-5.
40. Jackson, L., et al., Adult mesenchymal stem cells: differentiation potential and therapeutic applications. J Postgrad Med, 2007. 53(2): p. 121-7.
41. Pittenger, M. F., et al., Multilineage potential of adult human mesenchymal stem cells. Science, 1999. 284(5411): p. 143-7.
42. Banas, A., et al., Rapid hepatic fate specification of adipose-derived stem cells and their therapeutic potential for liver failure. J Gastroenterol Hepatol, 2009. 24(1): p. 70-7.
43. Lee, K. D., et al., In vitro hepatic differentiation of human mesenchymal stem cells. Hepatology, 2004. 40(6): p. 1275-84.
44. Cho, K. A., et al., Mesenchymal stem cells showed the highest potential for the regeneration of injured liver tissue compared with other subpopulations of the bone marrow. Cell Biol Int, 2009. 33(7): p. 772-7.
45. Hong, S. H., et al., In vitro differentiation of human umbilical cord blood-derived mesenchymal stem cells into hepatocyte-like cells. Biochem Biophys Res Commun, 2005. 330(4): p. 1153-61.
46. Ishikawa, T., et al., Stem cells for hepatic regeneration: the role of adipose tissue derived mesenchymal stem cells. Curr Stem Cell Res Ther, 2010. 5(2): p. 182-9.
47. Seo, M. J., et al., Differentiation of human adipose stromal cells into hepatic lineage in vitro and in vivo. Biochem Biophys Res Commun, 2005. 328(1): p. 258-64.
48. Crisan, M., et al., A perivascular origin for mesenchymal stem cells in multiple human organs. Cell Stem Cell, 2008. 3(3): p. 301-13.
49. Tavian, M. and B. Peault, Embryonic development of the human hematopoietic system. Int J Dev Biol, 2005. 49(2-3): p. 243-50.
50. Peault, B., et al., Stem and progenitor cells in skeletal muscle development, maintenance, and therapy. Mol Ther, 2007. 15(5): p. 867-77.
51. Aggarwal, S. and M. F. Pittenger, Human mesenchymal stem cells modulate allogeneic immune cell responses. Blood, 2005. 105(4): p. 1815-22.
52. Caplan, A. I., Adult mesenchymal stem cells for tissue engineering versus regenerative medicine. J Cell Physiol, 2007. 213(2): p. 341-7.
53. Chamberlain, G., et al., Concise review: mesenchymal stem cells: their phenotype, differentiation capacity, immunological features, and potential for homing. Stem Cells, 2007. 25(11): p. 2739-49.
54. Banas, A., et al., IFATS collection: in vivo therapeutic potential of human adipose tissue mesenchymal stem cells after transplantation into mice with liver injury. Stem Cells, 2008. 26(10): p. 2705-12.
55. Kharaziha, P., et al., Improvement of liver function in liver cirrhosis patients after autologous mesenchymal stem cell injection: a phase I-II clinical trial. Eur J Gastroenterol Hepatol, 2009. 21(10): p. 1199-205.
56. Kuo, T. K., et al., Stem cell therapy for liver disease: parameters governing the success of using bone marrow mesenchymal stem cells. Gastroenterology, 2008. 134(7): p. 2111-21, 2121 e1-3.
57. Chang, Y. J., et al., Mesenchymal stem cells facilitate recovery from chemically induced liver damage and decrease liverfibrosis. Life Sci, 2009. 85(13-14): p. 517-25.
58. Lu, L. L., et al., Isolation and characterization of human umbilical cord mesenchymal stem cells with hematopoiesis-supportive function and other potentials. Haematologica, 2006. 91(8): p. 1017-26.
59. Mohamadnejad, M., et al., Phase 1 trial of autologous bone marrow mesenchymal stem cell transplantation in patients with decompensated liver cirrhosis. Arch Iran Med, 2007. 10(4): p. 459-66.
60. Terai, S., et al., Improved liver function in patients with liver cirrhosis after autologous bone marrow cell infusion therapy. Stem Cells, 2006. 24(10): p. 2292-8.
61. Chang, C. J., et al., Placenta-derived multipotent cells exhibit immunosuppressive properties that are enhanced in the presence of interferon-gamma. Stem Cells, 2006. 24(11): p. 2466-77.
62. Iyer, S. S. and M. Rojas, Anti-inflammatory effects of mesenchymal stem cells: novel concept for future therapies. Expert Opin Biol Ther, 2008. 8(5): p. 569-81.
63. Nauta, A. J. and W. E. Fibbe, Immunomodulatory properties of mesenchymal stromal cells. Blood, 2007. 110(10): p. 3499-506.
64. Uccelli, A., V. Pistoia, and L. Moretta, Mesenchymal stem cells: a new strategy for immunosuppression? Trends Immunol, 2007. 28(5): p. 219-26.
65. Wolbank, S., et al., Dose-dependent immunomodulatory effect of human stem cells from amniotic membrane: a comparison with human mesenchymal stem cells from adipose tissue. Tissue Eng, 2007. 13(6): p. 1173-83.
66. Wolf, D. and A. M. Wolf, Mesenchymal stem cells as cellular immunosuppressants. Lancet, 2008. 371(9624): p. 1553-4.
67. Shi, M., Z. W. Liu, and F. S. Wang, Immunomodulatory properties and therapeutic application of mesenchymal stem cells. Clin Exp Immunol, 2011. 164(1): p. 1-8.
68. Sordi, V. and L. Piemonti, Therapeutic plasticity of stem cells and allograft tolerance. Cytotherapy, 2011. 13(6): p. 647-60.
69. Popp, F. C., et al., Mesenchymal stem cells as immunomodulators after liver transplantation. Liver Transpl, 2009. 15(10): p. 1192-8.
70. Friedenstein, A. J., R. K. Chailakhjan, and K. S. Lalykina, The development of fibroblast colonies in monolayer cultures of guinea-pig bone marrow and spleen cells. Cell Tissue Kinet, 1970. 3(4): p. 393-403.
71. Friedenstein, A. J., et al., Stromal cells responsible for transferring the microenvironment of the hemopoietic tissues. Cloning in vitro and retransplantation in vivo. Transplantation, 1974. 17(4): p. 331-40.
72. Caplan, A. I., Molecular and cellular differentiation of muscle, cartilage, and bone in the developing limb. Prog Clin Biol Res, 1986. 217B: p. 307-18.
73. Caplan, A. I., Mesenchymal stem cells. J Orthop Res, 1991. 9(5): p. 641-50.
74. Kopen, G. C., D. J. Prockop, and D. G. Phinney, Marrow stromal cells migrate throughout forebrain and cerebellum, and they differentiate into astrocytes after injection into neonatal mouse brains. Proc Natl Acad Sci USA, 1999. 96(19): p. 10711-6.
75. Teratani, T., et al., Direct hepatic fate specification from mouse embryonic stem cells. Hepatology, 2005. 41(4): p. 836-46.
76. Chamberlain, J., et al., Efficient generation of human hepatocytes by the intrahepatic delivery of clonal human mesenchymal stem cells in fetal sheep. Hepatology, 2007. 46(6): p. 1935-45.
77. Gao, J., et al., The dynamic in vivo distribution of bone marrow-derived mesenchymal stem cells after infusion. Cells Tissues Organs, 2001. 169(1): p. 12-20.
78. Le Blanc, K. and O. Ringden, Immunomodulation by mesenchymal stem cells and clinical experience. J Intern Med, 2007. 262(5): p. 509-25.
79. Keyser, K. A., K. E. Beagles, and H. P. Kiem, Comparison of mesenchymal stem cells from different tissues to suppress T-cell activation. Cell Transplant, 2007. 16(5): p. 555-62.
80. Le Blanc, K., et al., Mesenchymal stem cells for treatment of steroid-resistant, severe, acute graft-versus-host disease: a phase II study. Lancet, 2008. 371(9624): p. 1579-86.
81. Ning, H., et al., The correlation between cotransplantation of mesenchymal stem cells and higher recurrence rate in hematologic malignancy patients: outcome of a pilot clinical study. Leukemia, 2008. 22(3): p. 593-9.
82. Ball, L., et al., Third party mesenchymal stromal cell infusions fail to induce tissue repair despite successful control of severe grade IV acute graft-versus-host disease in a child with juvenile myelo-monocytic leukemia. Leukemia, 2008. 22(6): p. 1256-7.
83. Ringden, O., et al., Mesenchymal stem cells for treatment of therapy-resistant graft-versus-host disease. Transplantation, 2006. 81(10): p. 1390-7.
84. Le Blanc, K., et al., Treatment of severe acute graft-versus-host disease with third party haploidentical mesenchymal stem cells. Lancet, 2004. 363(9419): p. 1439-41.
85. Muller, I., et al., Application of multipotent mesenchymal stromal cells in pediatric patients following allogeneic stem cell transplantation. Blood Cells Mol Dis, 2008. 40(1): p. 25-32.
86. Horwitz, E. M., et al., Isolated allogeneic bone marrow-derived mesenchymal cells engraft and stimulate growth in children with osteogenesis imperfecta: Implications for cell therapy of bone. Proc Natl Acad Sci USA, 2002. 99(13): p. 8932-7.
87. Koc, O. N., et al., Allogeneic mesenchymal stem cell infusion for treatment of metachromatic leukodystrophy (MLD) and Hurler syndrome (MPS-IH). Bone Marrow Transplant, 2002. 30(4): p. 215-22.
88. Le Blanc, K., et al., Transplantation of mesenchymal stem cells to enhance engraftment of hematopoietic stem cells. Leukemia, 2007. 21(8): p. 1733-8.
89. Lazarus, H. M., et al., Cotransplantation of HLA-identical sibling culture-expanded mesenchymal stem cells and hematopoietic stem cells in hematologic malignancy patients. Biol Blood Marrow Transplant, 2005. 11(5): p. 389-98.
90. Ball, L. M., et al., Cotransplantation of ex vivo expanded mesenchymal stem cells accelerates lymphocyte recovery and may reduce the risk of graft failure in haploidentical hematopoietic stem-cell transplantation. Blood, 2007. 110(7): p. 2764-7.
91. http://www.osiristx.com/pdf/PR %2039%2025Mar07%20Provacel %20Positive %2 0Results.pdf.
92. Kurtzberg, J., et al., Allogeneic human mesenchymal stem cell therapy (remestemcel-L, Prochymal) as a rescue agent for severe refractory acute graft-versus-host disease in pediatric patients. Biol Blood Marrow Transplant, 2014. 20(2): p. 229-35.
93. Kellathur, S. N. and H. X. Lou, Cell and tissue therapy regulation: worldwide status and harmonization. Biologicals, 2012. 40(3): p. 222-4.
94. Chen, Y., et al., In vitro differentiation of mouse bone marrow stromal stem cells into hepatocytes induced by conditioned culture medium of hepatocytes. J Cell Biochem, 2007. 102(1): p. 52-63.
95. Mohsin, S., et al., Enhanced hepatic differentiation of mesenchymal stem cells after pretreatment with injured liver tissue. Differentiation, 2011. 81(1): p. 42-8.
96. Li, W., H. Jiang, and J. M. Feng, Isogenic mesenchymal stem cells transplantation improves a rat model of chronic aristolochic acid nephropathy via upregulation of hepatic growth factor and downregulation of transforming growth factor beta1. Mol Cell Biochem, 2012. 368(1-2): p. 137-45.
97. Soleymaninejadian, E., K. Pramanik, and E. Samadian, Immunomodulatory properties of mesenchymal stem cells: cytokines and factors. Am J Reprod Immunol, 2012. 67(1): p. 1-8.
98. Lange, C., et al., Hepatocytic gene expression in cultured rat mesenchymal stem cells. Transplant Proc, 2005. 37(1): p. 276-9.
99. Li, Z., et al., In vitro and in vivo characteristics of hepatic oval cells modified with human hepatocyte growth factor. Cell Mol Biol Lett, 2013. 18(4): p. 507-21.
100. Hua, Z. Y., et al., The effect of hepatocyte growth factor on the initiation phase of liver regeneration after cold ischemia in a rat model of small-for-size liver transplantation. Hepatogastroenterology, 2012. 59(117): p. 1548-52.
101. Lee, Y. J., et al., Apoptotic cell instillation after bleomycin attenuates lung injury through hepatocyte growth factor induction. Eur Respir J, 2012. 40(2): p. 424-35.
102. Kaldenbach, M., et al., Hepatocyte growth factor/c-Met signalling is important for the selection of transplanted hepatocytes. Gut, 2012. 61(8): p. 1209-18.
103. Kroy, D. C., et al., Hepatocyte specific deletion of c-Met leads to the development of severe non-alcoholic steatohepatitis in mice. J Hepatol, 2014.
104. Francois, S., et al., Human mesenchymal stem cells provide protection against radiation-induced liver injury by antioxidative process, vasculature protection, hepatocyte differentiation, and trophic effects. Biomed Res Int, 2013. 2013: p. 151679.
105. Enriquez-Cortina, C., et al., Hepatocyte growth factor protects against isoniazid/rifampicin-induced oxidative liver damage. Toxicol Sci, 2013. 135(1): p. 26-36.
106. Lee, Y. H., et al., Hepatocyte growth factor inhibits apoptosis by the profibrotic factor angiotensin II via extracellular signal-regulated kinase ½ in endothelial cells and tissue explants. Mol Biol Cell, 2010. 21(23): p. 4240-50.
107. Fang, B., et al., Systemic infusion of FLK1(+) mesenchymal stem cells ameliorate carbon tetrachloride-induced liver fibrosis in mice. Transplantation, 2004. 78(1): p. 83-8.
108. Zhao, W., et al., Intravenous injection of mesenchymal stem cells is effective in treating liver fibrosis. World J Gastroenterol, 2012. 18(10): p. 1048-58.
109. Miyazaki, M., et al., Isolation of a bone marrow-derived stem cell line with high proliferation potential and its application for preventing acute fatal liver failure. Stem Cells, 2007. 25(11): p. 2855-63.
110. Kaibori, M., et al., Bone marrow cells enhance liver regeneration after massive hepatectomy in mice. Dig Dis Sci, 2014. 59(7): p. 1484-9.
111. Ezquer, M., et al., Intravenous administration of multipotent stromal cells prevents the onset of non-alcoholic steatohepatitis in obese mice with metabolic syndrome. J Hepatol, 2011. 55(5): p. 1112-20.
112. Hwang, S., et al., Hepatogenic differentiation of mesenchymal stem cells in a rat model of thioacetamide-induced liver cirrhosis. Cell Biol Int, 2012. 36(3): p. 279-88.
113. Xu, J., et al., The types of hepatic myofibroblasts contributing to liver fibrosis of different etiologies. Front Pharmacol, 2014. 5: p. 167.
114. Novo, E., et al., Cellular and molecular mechanisms in liver fibrogenesis. Arch Biochem Biophys, 2014. 548: p. 20-37.
115. Wu, Z., et al., Pseudoephedrine/ephedrine shows potent anti-inflammatory activity against TNF-alpha-mediated acute liver failure induced by lipopolysaccharide/D-galactosamine. Eur J Pharmacol, 2014. 724: p. 112-21.
116. Lindros, K. O. and H. A. Jarvelainen, Chronic systemic endotoxin exposure: an animal model in experimental hepatic encephalopathy. Metab Brain Dis, 2005. 20(4): p. 393-8.
117. Sandler, N. G., et al., Host response to translocated microbial products predicts outcomes ofpatients with HBV or HCV infection. Gastroenterology, 2011. 141(4): p. 1220-30, 1230 e1-3.
118. Yuan, S., et al., The role of bone marrow mesenchymal stem cells in the treatment of acute liver failure. Biomed Res Int, 2013. 2013: p. 251846.
119. Tekkesin, N., et al., Induction of HGF and VEGF in hepatic regeneration after hepatotoxin-induced cirrhosis in mice. Hepatogastroenterology, 2011. 58(107-108): p. 971-9.
120. Kajdaniuk, D., et al., Vascular endothelial growth factor (VEGF) -part 1: in physiology and pathophysiology. Endokrynol Pol, 2011. 62(5): p. 444-55.
121. Ueno, T., et al., Angiogenic cell therapy for hepatic fibrosis. Med Mol Morphol, 2006. 39(1): p. 16-21.
122. Sturm, J., et al., Liver regeneration in FGF-2-deficient mice: VEGF acts as potential functional substitute for FGF-2. Liver Int, 2004. 24(2): p. 161-8.
123. Sun, L., et al., Bone mesenchymal stem cell transplantation via four routes for the treatment of acute liver failure in rats. Int J Mol Med, 2014.
124. Peng, L., et al., Autologous bone marrow mesenchymal stem cell transplantation in liver failure patients caused by hepatitis B: short-term and long-term outcomes. Hepatology, 2011. 54(3): p. 820-8.
125. Xie, C., et al., Human bone marrow mesenchymal stem cells are resistant to HBV infection during differentiation into hepatocytes in vivo and in vitro. Cell Biol Int, 2009. 33(4): p. 493-500.
126. Mohamadnejad, M., et al., Randomized placebo-controlled trial of mesenchymal stem cell transplantation in decompensated cirrhosis. Liver Int, 2013. 33(10): p. 1490-6.
127. Jang, Y. O., et al., Histological improvement following administration of autologous bone marrow-derived mesenchymal stem cells for alcoholic cirrhosis: a pilot study. Liver Int, 2014. 34(1): p. 33-41.
128. Amer, M. E., et al., Clinical and laboratory evaluation of patients with end-stage liver cell failure injected with bone marrow-derived hepatocyte-like cells. Eur J Gastroenterol Hepatol, 2011. 23(10): p. 936-41.
129. Walter, D. H., et al., Impaired CXCR4 signaling contributes to the reduced neovascularization capacity of endothelial progenitor cells from patients with coronary artery disease. Circ Res, 2005. 97(11): p. 1142-51.
130. Cipriani, P., et al., Impairment of endothelial cell differentiation from bone marrow-derived mesenchymal stem cells: new insight into the pathogenesis of systemic sclerosis. Arthritis Rheum, 2007. 56(6): p. 1994-2004.
131. Rodriguez-Menocal, L., et al., Stimulation of skin and wound fibroblast migration by mesenchymal stem cells derived from normal donors and chronic wound patients. Stem Cells Transl Med, 2012. 1(3): p. 221-9.
132. Sugihara, S., et al., Age-related BM-MNC dysfunction hampers neovascularization. Mech Ageing Dev, 2007. 128(9): p. 511-6.
133. Zhuo, Y., et al., Aging impairs the angiogenic response to ischemic injury and the activity of implanted cells: combined consequences for cell therapy in older recipients. J Thorac Cardiovasc Surg, 2010. 139(5): p. 1286-94, 1294 e1-2.
134. Teraa, M., et al., Bone marrow alterations and lower endothelial progenitor cell numbers in critical limb ischemia patients. PLoS One, 2013. 8(1): p. e55592.
135. Bozdag-Turan, I., et al., Correlation between the functional impairment of bone marrow-derived circulating progenitor cells and the extend of coronary artery disease. J Transl Med, 2012. 10: p. 143.
136. Puglisi, M. A., et al., Adipose tissue-derived mesenchymal stem cells and hepatic differentiation: old concepts and future perspectives. Eur Rev Med Pharmacol Sci, 2011. 15(4): p. 355-64.
137. Strioga, M., et al., Same or not the same? Comparison of adipose tissue-derived versus bone marrow-derived mesenchymal stem and stromal cells. Stem Cells Dev, 2012. 21(14): p. 2724-52.
138. Yi, T. and S. U. Song, Immunomodulatory properties of mesenchymal stem cells and their therapeutic applications. Arch Pharm Res, 2012. 35(2): p. 213-21.
139. Mosna, F., L. Sensebe, and M. Krampera, Human bone marrow and adipose tissue mesenchymal stem cells: a user's guide. Stem Cells Dev, 2010. 19(10): p. 1449-70.
140. Zuk, P. A., et al., Multilineage cells from human adipose tissue: implications for cell-based therapies. Tissue Eng, 2001. 7(2): p. 211-28.
141. Zuk, P. A., et al., Human adipose tissue is a source of multipotent stem cells. Mol Biol Cell, 2002. 13(12): p. 4279-95.
142. Ong, W. K. and S. Sugii, Adipose-derived stem cells: fatty potentials for therapy. Int J Biochem Cell Biol, 2013. 45(6): p. 1083-6.
143. Bassi, G., et al., Adipose-derived stromal cells (ASCs). Transfus Apher Sci, 2012. 47(2): p. 193-8.
144. Tallone, T., et al., Adult human adipose tissue contains several types of multipotent cells. J Cardiovasc Transl Res, 2011. 4(2): p. 200-10.
145. Garcia-Olmo, D., et al., A phase I clinical trial of the treatment of Crohn's fistula by adipose mesenchymal stem cell transplantation. Dis Colon Rectum, 2005. 48(7): p. 1416-23.
146. Stillaert, F. B., et al., Human clinical experience with adipose precursor cells seeded on hyaluronic acid-based spongy scaffolds. Biomaterials, 2008. 29(29): p. 3953-9.
147. Garcia-Olmo, D., M. Garcia-Arranz, and D. Herreros, Expanded adipose-derived stem cells for the treatment of complex perianal fistula including Crohn's disease. Expert Opin Biol Ther, 2008. 8(9): p. 1417-23.
148. Fang, B., et al., Treatment of severe therapy-resistant acute graft-versus-host disease with human adipose tissue-derived mesenchymal stem cells. Bone Marrow Transplant, 2006. 38(5): p. 389-90.
149. Fang, B., et al., Using human adipose tissue-derived mesenchymal stem cells as salvage therapy for hepatic graft-versus-host disease resembling acute hepatitis. Transplant Proc, 2007. 39(5): p. 1710-3.
150. Fang, B., et al., Favorable response to human adipose tissue-derived mesenchymal stem cells in steroid-refractory acute graft-versus-host disease. Transplant Proc, 2007. 39(10): p. 3358-62.
151. Hayashi, O., et al., Comparison of osteogenic ability of rat mesenchymal stem cells from bone marrow, periosteum, and adipose tissue. Calcif Tissue Int, 2008. 82(3): p. 238-47.
152. Noel, D., et al., Cell specific differences between human adipose-derived and mesenchymal-stromal cells despite similar differentiation potentials. Exp Cell Res, 2008. 314(7): p. 1575-84.
153. Kim, Y., et al., Direct comparison of human mesenchymal stem cells derived from adipose tissues and bone marrow in mediating neovascularization in response to vascular ischemia. Cell Physiol Biochem, 2007. 20(6): p. 867-76.
154. Lin, C. S., G. Lin, and T. F. Lue, Allogeneic and xenogeneic transplantation of adipose-derived stem cells in immunocompetent recipients without immunosuppressants. Stem Cells Dev, 2012. 21(15): p. 2770-8.
155. Yukawa, H., et al., Cell transplantation of adipose tissue-derived stem cells in combination with heparin attenuated acute liver failure in mice. Cell Transplant, 2009. 18(5): p. 611-8.
156. Yukawa, H., et al., Monitoring transplanted adipose tissue-derived stem cells combined with heparin in the liver by fluorescence imaging using quantum dots. Biomaterials, 2012. 33(7): p. 2177-86.
157. Kim, S. J., et al., Therapeutic potential of adipose tissue-derived stem cells for liver failure according to the transplantation routes. J Korean Surg Soc, 2011. 81(3): p. 176-86.
158. Deng, L., et al., Adipose derived mesenchymal stem cells efficiently rescue carbon tetrachloride-induced acute liver failure in mouse. ScientificWorldJournal, 2014. 2014: p. 103643.
159. Salomone, F., et al., Efficacy of adipose tissue-mesenchymal stem cell transplantation in rats with acetaminophen liver injury. Stem Cell Res, 2013. 11(3): p. 1037-44.
160. Avital, I., et al., Bone marrow-derived liver stem cell and mature hepatocyte engraftment in livers undergoing rejection. Surgery, 2002. 132(2): p. 384-90.
161. Tateishi-Yuyama, E., et al., Therapeutic angiogenesis for patients with limb ischaemia by autologous transplantation of bone-marrow cells: a pilot study and a randomised controlled trial. Lancet, 2002. 360(9331): p. 427-35.
162. Nizankowski, R., et al., The treatment of advanced chronic lower limb ischaemia with marrow stem cell autotransplantation. Kardiol Pol, 2005. 63(4): p. 351-60; discussion 361.
163. Fernandez-Aviles, F., et al., Experimental and clinical regenerative capability of human bone marrow cells after myocardial infarction. Circ Res, 2004. 95(7): p. 742-8.
164. Iihoshi, S., et al., A therapeutic window for intravenous administration of autologous bone marrow after cerebral ischemia in adult rats. Brain Res, 2004. 1007(1-2): p. 1-9.
165. Strauer, B. E., et al., Repair of infarcted myocardium by autologous intracoronary mononuclear bone marrow cell transplantation in humans. Circulation, 2002. 106(15): p. 1913-8.
166. de Vasconcelos Dos Santos, A., et al., Therapeutic window for treatment of cortical ischemia with bone marrow-derived cells in rats. Brain Res, 2010. 1306: p. 149-58.
167. Terai, S., et al., An in vivo model for monitoring trans-differentiation of bone marrow cells into functional hepatocytes. J Biochem, 2003. 134(4): p. 551-8.
168. Sakaida, I., et al., Transplantation of bone marrow cells reduces CCl4-induced liver fibrosis in mice. Hepatology, 2004. 40(6): p. 1304-11.
169. Bellayr, I. H., X. Mu, and Y. Li, Biochemical insights into the role of matrix metalloproteinases in regeneration: challenges and recent developments. Future Med Chem, 2009. 1(6): p. 1095-1111.
170. Malemud, C. J., Matrix metalloproteinases (MMPs) in health and disease: an overview. Front Biosci, 2006. 11: p. 1696-701.
171. Kawai, K., et al., Matrix metalloproteinase-9 contributes to the mobilization of bone marrow cells in the injured liver. Cell Transplant, 2012. 21(2-3): p. 453-64.
172. Jiao, J., et al., Dendritic cell regulation of carbon tetrachloride-induced murine liver fibrosis regression. Hepatology, 2012. 55(1): p. 244-55.
173. Handgretinger, R. and S. Kuci, CD133-Positive Hematopoietic Stem Cells: From Biology to Medicine. Adv Exp Med Biol, 2013. 777: p. 99-111.
174. Zocco, M. A., et al., CD133+ stem cell mobilization after partial hepatectomy depends on resection extent and underlying disease. Dig Liver Dis, 2011. 43(2): p. 147-54.
175. Harb, R., et al., Bone marrow progenitor cells repair rat hepatic sinusoidal endothelial cells after liver injury. Gastroenterology, 2009. 137(2): p. 704-12.
176. Gehling, U. M., et al., Partial hepatectomy induces mobilization of a unique population of haematopoietic progenitor cells in human healthy liver donors. J Hepatol, 2005. 43(5): p. 845-53.
177. Rountree, C. B., et al., A CD133-expressing murine liver oval cell population with bilineage potential. Stem Cells, 2007. 25(10): p. 2419-29.
178. Rountree, C. B., et al., Isolation of CD133+ liver stem cells for clonal expansion. J Vis Exp, 2011(56).
179. Yovchev, M. I., et al., Novel hepatic progenitor cell surface markers in the adult rat liver. Hepatology, 2007. 45(1): p. 139-49.
180. am Esch, J. S., 2nd, et al., Portal application of autologous CD133+ bone marrow cells to the liver: a novel concept to support hepatic regeneration. Stem Cells, 2005. 23(4): p. 463-70.
181. Sidney, L. E., et al., Concise review: evidence for CD34 as a common marker for diverse progenitors. Stem Cells, 2014. 32(6): p. 1380-9.
182. Crosby, H. A., D. A. Kelly, and A. J. Strain, Human hepatic stem-like cells isolated using c-kit or CD34 can differentiate into biliary epithelium. Gastroenterology, 2001. 120(2): p. 534-44.
183. Theise, N. D., et al., Derivation of hepatocytes from bone marrow cells in mice after radiation-induced myeloablation. Hepatology, 2000. 31(1): p. 235-40.
184. Gordon, M. Y., et al., Characterization and clinical application of human CD34+ stem/progenitor cell populations mobilized into the blood by granulocyte colony-stimulating factor. Stem Cells, 2006. 24(7): p. 1822-30.
185. Levicar, N., et al., Long-term clinical results of autologous infusion of mobilized adult bone marrow derived CD34+ cells in patients with chronic liver disease. Cell Prolif, 2008. 41 Suppl 1: p. 115-25.
186. Spahr, L., et al., Autologous bone marrow mononuclear cell transplantation in patients with decompensated alcoholic liver disease: a randomized controlled trial. PLoS One, 2013. 8(1): p. e53719.
187. Bai, Y. Q., et al., Outcomes of autologous bone marrow mononuclear cell transplantation in decompensated liver cirrhosis. World J Gastroenterol, 2014. 20(26): p. 8660-6.
188. Lyra, A. C., et al., Feasibility and safety of autologous bone marrow mononuclear cell transplantation in patients with advanced chronic liver disease. World J Gastroenterol, 2007. 13(7): p. 1067-73.
189. Lyra, A. C., et al., Infusion of autologous bone marrow mononuclear cells through hepatic artery results in a short-term improvement of liver function in patients with chronic liver disease: a pilot randomized controlled study. Eur J Gastroenterol Hepatol, 2010. 22(1): p. 33-42.
190. Cantz, T., et al., Reevaluation of bone marrow-derived cells as a source for hepatocyte regeneration. Cell Transplant, 2004. 13(6): p. 659-66.
191. Jang, Y. Y., et al., Hematopoietic stem cells convert into liver cells within days without fusion. Nat Cell Biol, 2004. 6(6): p. 532-9.
192. Kanazawa, Y. and I. M. Verma, Little evidence of bone marrow-derived hepatocytes in the replacement of injured liver. Proc Natl Acad Sci USA, 2003. 100 Suppl 1: p. 11850-3.
193. Shizhu, J., et al., Bone marrow mononuclear cell transplant therapy in mice with CCl4-induced acute liver failure. Turk J Gastroenterol, 2012. 23(4): p. 344-52.
194. Lee, M. J., et al., Anti-fibrotic effect of chorionic plate-derived mesenchymal stem cells isolated from human placenta in a rat model of CCl(4)-injured liver: potential application to the treatment of hepatic diseases. J Cell Biochem, 2010. 111(6): p. 1453-63.
195. Cao, H., et al., Therapeutic potential of transplanted placental mesenchymal stem cells in treating Chinese miniature pigs with acute liver failure. BMC Med, 2012. 10: p. 56.
196. Shi, M., et al., Human mesenchymal stem cell transfusion is safe and improves liver function in acute-on-chronic liver failure patients. Stem Cells Transl Med, 2012. 1(10): p. 725-31.
197. Gridelli, B., et al., Efficient human fetal liver cell isolation protocol based on vascular perfusion for liver cell-based therapy and case report on cell transplantation. Liver Transpl, 2012. 18(2): p. 226-37.
198. Ahmadi, M., et al., Bone marrow mesenchymal stem cells and their conditioned media could potentially ameliorate ovalbumin-induced asthmatic changes. Biomed Pharmacother, 2016. 85: p. 28-40.
199. Nemunaitis, J., et al., Human marrow stromal cells: response to interleukin-6 (IL-6) and control of IL-6 expression. Blood, 1989. 74(6): p. 1929-35.
200. Sadovnikova, E. Y., et al., Induction of hematopoietic microenvironment by the extracellular matrix from long-term bone marrow cultures. Ann Hematol, 1991. 62(5): p. 160-4.
201. Lazarus, H. M., et al., Ex vivo expansion and subsequent infusion of human bone marrow-derived stromal progenitor cells (mesenchymal progenitor cells): implications for therapeutic use. Bone Marrow Transplant, 1995. 16(4): p. 557-64.
202. Yoo, J. U., et al., The chondrogenic potential of human bone-marrow-derived mesenchymal progenitor cells. J Bone Joint Surg Am, 1998. 80(12): p. 1745-57.
203. Fleming, J. E., Jr., et al., Monoclonal antibody against adult marrow-derived mesenchymal stem cells recognizes developing vasculature in embryonic human skin. Dev Dyn, 1998. 212(1): p. 119-32.
204. Ghilzon, R., C. A. McCulloch, and R. Zohar, Stromal mesenchymal progenitor cells. Leuk Lymphoma, 1999. 32(3-4): p. 211-21.
205. De Cesaris, V., et al., Isolation, proliferation and characterization of endometrial canine stem cells. Reprod Domest Anim, 2016.
206. Van Pham, P., et al., Isolation and proliferation of umbilical cord tissue derived mesenchymal stem cells for clinical applications. Cell Tissue Bank, 2016. 17(2): p. 289-302.
207. Zhao, G., et al., Large-scale expansion of Wharton's jelly-derived mesenchymal stem cells on gelatin microbeads, with retention of self-renewal and multipotency characteristics and the capacity for enhancing skin wound healing. Stem Cell Res Ther, 2015. 6: p. 38.
208. Huang, P., et al., Differentiation of human umbilical cord Wharton's jelly-derived mesenchymal stem cells into germ-like cells in vitro. J Cell Biochem, 2010. 109(4): p. 747-54.
209. Wang, S. J., et al., Chondrogenic Potential of Peripheral Blood Derived Mesenchymal Stem Cells Seeded on Demineralized Cancellous Bone Scaffolds. Sci Rep, 2016. 6: p. 36400.
210. Fazeli, Z., M. D. Omrani, and S. M. Ghaderian, CD29/CD184 expression analysis provides a signature for identification of neuronal like cells differentiated from PBMSCs. Neurosci Lett, 2016. 630: p. 189-93.
211. Wu, G., et al., Osteogenesis of peripheral blood mesenchymal stem cells in self assembling peptide nanofiber for healing critical size calvarial bony defect. Sci Rep, 2015. 5: p. 16681.
212. Shaer, A., et al., Isolation and characterization of Human Mesenchymal Stromal Cells Derived from Placental Decidua Basalis; Umbilical cord Wharton's Jelly and Amniotic Membrane. Pak J Med Sci, 2014. 30(5): p. 1022-6.
213. Fu, W. L., C. Y. Zhou, and J. K. Yu, A new source of mesenchymal stem cells for articular cartilage repair: MSCs derived from mobilized peripheral blood share similar biological characteristics in vitro and chondrogenesis in vivo as MSCs from bone marrow in a rabbit model. Am J Sports Med, 2014. 42(3): p. 592-601.
214. El-Badawy, A., et al., Adipose Stem Cells Display Higher Regenerative Capacities and More Adaptable Electro-Kinetic Properties Compared to Bone Marrow-Derived Mesenchymal Stromal Cells. Sci Rep, 2016. 6: p. 37801.
215. Plock, J. A., et al., The Influence of Timing and Frequency of Adipose-Derived Mesenchymal Stem Cell Therapy on Immunomodulation Outcomes After Vascularized Composite Allotransplantation. Transplantation, 2017. 101(1): p. e1-e11.

1. A method of preventing, and/or inhibiting, and/or reversing liver failure comprising the steps of:
a) identifying a patient suffering from liver failure;
b) extracting immune cells from a subject;
c) contacting said immune cells with regenerative cells in a manner so that regenerative cells endow onto said immune cells properties capable of inhibiting and/or reversing liver failure; and
d) administering said immune cells into said patient.

2. The method of claim 1, wherein said liver failure is associated with fibrosis.

3. The method of claim 1, wherein said liver failure is associated with alcoholism.

4. The method of claim 1, wherein said liver failure is associated with viral damage.

5. The method of claim 1, wherein said liver failure is associated with inflammation.

6. The method of claim 1, wherein said liver failure is non-alcoholic steatohepatitis.

7. The method of claim 1, wherein said liver failure is autoimmune mediated.

8. The method of claim 1, wherein said subject is not the patient.

9. The method of claim 8, wherein said immune cells are xenogeneic.

10. The method of claim 8, wherein said immune cells are cord blood derived.

11. The method of claim 8, wherein said immune cells are derived from pluripotent stem cells.

12. The method of claim 1, wherein said immune cells are cultured together with said regenerative cells in the presence of an activator of an immune receptor.

13. The method of claim 12, wherein said immune receptor activates immunotyrosine activation motifs.

14. The method of claim 12, wherein said immune receptor activates NF-AT.

15. The method of claim 12, wherein said immune receptor activates NF-kappa B.

16. The method of claim 12, wherein said immune receptor activates STAT-3.

17. The method of claim 12, wherein said immune receptor activates janus activated kinase.

18. The method of claim 12, wherein said immune receptor activates MAP-kinase.

19. The method of claim 12, wherein said immune receptor is TLR. 1

20. The method of claim 19, wherein said TLR-1 is activated by Pam3CSK4.
